(19) United States
(12) Patent Application Publication
(10) Pub. No.: US 2025/0000847 A1
(43) Pub. Date: Jan. 2, 2025

(54) TREATMENT OF NEURODEGENERATIVE DISEASES WITH HDAC INHIBITORS

(71) Applicant: CASE WESTERN RESERVE UNIVERSITY, Cleavland', OH (US)

(72) Inventors: Paul J. Tesar, Wickliffe, OH (US); Benjamin L.L. Clayton, University Heights, OH (US)

(21) Appl. No.: 18/687,441

(22) PCT Filed: Aug. 31, 2022
(86) PCT No.: PCT/US2022/042242

§ 371 Date: Feb. 28, 2024

Related U.S. Application Data
(60) Provisional application No. 63/239,641, filed on Sep. 1, 2021.

(51) Int. Cl. A61K 31/415; A61K 31/167; A61K 31/4192; A61P 25/00

(52) U.S. Cl. CPC A61K 31/415; A61K 31/167; A61K 31/4192; A61P 25/00

(57) ABSTRACT

The invention described herein provides methods and reagents for treating diseases or conditions associated with reactive astrocytes using HD AC inhibitors, such as HDAC3-specific inhibitors.

### REFERENCE TO RELATED APPLICATION

This application claims priority to and the benefit of the filing date of U.S. Provisional Patent Application No. 63/239,641, filed on Sep. 1, 2021, the entire contents of which, including any sequence listing and drawings, are incorporated herein by reference.

### GOVERNMENT SUPPORT

This invention was made with government support under Grant No. R35NS116842, awarded by the National Institute of Health and National Institute of Neurological Disorders and Stroke. The government has certain rights in the invention.

### BACKGROUND OF THE INVENTION

Astrocytes are stellate glial cells in the central nervous system (CNS) that are required for proper neuronal function. Astrocytes are the most abundant glial cell in the human brain, and readily respond to brain injury and play vital roles in the pathogenesis of neurodegenerative diseases (NDs). In response to brain injury including, inflammation, traumatic brain injury, stroke, and neurodegenerative diseases such as multiple sclerosis (MS), Alzheimer's disease (AD), Huntington's disease (HD), Parkinson's disease (PD), and amyotrophic lateral sclerosis (ALS), astrocyte can become polarized, and play either a beneficial or damaging role. Astrocytes that polarize to damaging reactive states contribute to brain damage, and represent a potential therapeutic target.

### SUMMARY OF THE INVENTION

One aspect of the invention provides a method for treating a subject with a disease or disorder associated with reactive astrocytes, comprising administering to the subject a therapeutically effective amount of one or more histone deacetylase (HDAC) inhibitors, wherein the one or more HDAC inhibitors suppress reactive astrocytes (e.g., suppress the formation, maintenance, and/or function of the reactive astrocytes).

In certain embodiments, the one or more HDAC inhibitors comprise a pan-HDAC inhibitor (such as AR-42, Belinostat, Givinostat, Dacinostat, M344, Panobinostat, Abexinostat, Pracinostat, Quisinostat, Rocilinostat, Scriptaid, Trichostatin A, and Vorinostat).

In certain embodiments, the one or more HDAC inhibitors target HDAC1, HDAC2, HDAC3, HDAC8, or a combination thereof. In certain embodiments, the one or more HDAC inhibitors are specific for HDAC3, such as RGFP966, BRD3308, or HDAC3-IN-T247 (T247). In certain embodiments, the HDAC inhibitor specific for HDAC3 is RGFP966.

In certain embodiments, the disease or disorder is one or more of disease or disorder of the brain, cerebral injury, brain and systemic disease, neurological disease, cerebral injury, disease associated with loss or reduction of level of calbindin, neurotoxicity, Niemann-Pick disease, Niemann-Pick Type A disease, Niemann-Pick Type B disease, Niemann-Pick Type C disease, neurodegenerative disorder, traumatic brain injury (TBI), autism, Alzheimer's, inflammatory disorder, neuroinflammatory disorder, neuroinflammation due to lysosomal storage disorder, lysosomal storage disorder, Gliobastoma multiforme, HIV, HIV associated cognitive deficits, brain tumor, disease responsive to treatment with histone deacetylase (HDAC) inhibitor, disease involving plasma concentration of vorinostat (SAHA), disease responsive to treatment with SAHA, disease where effect of SAHA is observed in animal model, encephalopathy, epilepsy, cerebrovascular disease, disease responsive to penetration of drug through the blood-brain barrier, Parkinson's disease, Amyotrophic Lateral Sclerosis, activator deficiency/GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, Gaucher's disease, Gaucher disease (types I-III), GM1 gangliosidosis, I-cell disease/muco lipidosis II, infantile free sialic acid storage disease/ISSD, juvenile hexosaminidase A deficiency, Krabbe disease, metachromatic leukodystrophy, mucopolysaccharidoses disorders, pseudo-Hurler polydystrophy/mucolipidosis IIIA, MPSI Hurler syndrome, MPSI Scheie syndrome, MPS I Hurler-Scheie syndrome, MPS II Hunter syndrome, Sanfilippo syndrome, Morquio syndrome, MPS FX hyaluronidase deficiency, MPS VI Maroteaux-Lamy, MPS VII Sly syndrome, mucolipidosis I/sialidosis, multiple sulfatase deficiency, neuronal ceroid lipofuscinoses, Pompe disease, Sandhoff disease, Schindler disease, Salla disease, Tay-Sachs spinal muscular atrophy, Huntington's disease, or a combination thereof.

In certain embodiments, the disease is selected from the group consisting of a neurodegenerative disease, a myelin or white matter disease, a genetic disease, an inflammatory disease or disorder, an acquired disease or disorder of the central nervous system, and a combination thereof.

In certain embodiments, the neurodegenerative disease is Alexander disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, diabetic neuropathy, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), wet or dry macular degeneration, Multiple System Atrophy, multiple sclerosis, Niemann Pick disease, Parkinson's disease, Pelizaeus-Merzbacher Disease, photoreceptor degenerative diseases such as retinitis pigmentosa and associated diseases, Pick's disease, primary lateral sclerosis, prion diseases, Progressive Supranuclear Palsy, Refsum's disease, Sandhoffs disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, or a combination thereof.

In certain embodiments, the myelin or white matter disease is acute disseminated encephalomyelitis, destructive leukoencephalopathy, leukoencephalitis, multiple sclerosis, cerebral palsy, periventricular leukomalacia (PVL), hypoxia induced white matter disease, neuromyelitis optica, adult-onset autosomal dominant leukodystrophy (ADLD), Aicardi-Goutieres syndrome, Alexander's disease, CADASIL, Canavan disease, CARASIL, cerebrotendinous xanthomatosis, childhood ataxia and cerebral hypomyelination (CACH)/vanishing white matter disease (VWMD), Fabry disease, fucosidosis, GM1 gangliosidosis, Krabbe's disease, L-2-hydroxyglutaric aciduria, megalencephalic leukoencephalopathy with subcortical cysts, metachromatic leukodystrophy, multiple sulfatase deficiency, Pelizaeus-Merzbacher disease, Pol III-Related Leukodystrophies, Refsum disease, salla disease (free sialic acid storage disease), Sjogren-Larsson syndrome, X-linked adrenoleukodystrophy, Zellweger syndrome spectrum disorders, or a combination thereof.

In certain embodiments, the genetic disease is one or more of Huntington's disease, adrenaleukodystrophy, Krabbe's disease, Pelizeaus-Merzbacher disease, vanishing white matter disease, Alexander's disease, metachromatic leukodystrophy, Megalencephalic Leukodystrophy with subcortical Cysts, Canavan's disease, lysosomal storage disorders, leukodystrophies, or a combination thereof.

In certain embodiments, the inflammatory disease or disorder is multiple sclerosis, neuromyelitis optica, chronic inflammatory demyelinating polyneuropathy, acute disseminated encephalomyelitis, acute optic neuritis, transverse myelitis, encephalitis, meningitis, or a combination thereof.

In certain embodiments, the acquired disease or disorder of the central nervous system is traumatic brain injury, chronic traumatic encephalopathy, stroke, brain metastasis, HIV associated cognitive impairment, neuroaids, cancer related cognitive impairment, immune effector cell-associated neurotoxicity syndrome (ICANS), or a combination thereof. In certain embodiments, the disease or disorder is Alzheimer's disease.

Another aspect of the invention provides a method of decreasing, in an subject in need thereof, reactive astrocyte induction/formation/conversion from resting astrocytes, the method comprising: administering to the subject an effective amount of one or more histone deacetylase (HDAC) inhibitors (such as HDAC3-specific inhibitor) to suppress reactive astrocytes; wherein the subject optionally is suspected of having, or at a high risk of having, a disease or disorder associated with reactive astrocytes, and/or has been identified as having reactive astrocytes.

In certain embodiments, the subject has been identified as having reactive astrocytes by comparing an image of the brain of the subject with a positive control brain image comprising reactive astrocytes, and a negative control brain image comprising non-reactive or resting astrocytes. In certain embodiments, the image of the brain of the subject is obtained by positron emission tomography (PET).

In certain embodiments, the reactive astrocyte induction/formation/conversion from resting astrocytes are determined to have decreased by comparing an image of the brain of the subject after administration of one or more histone deacetylase (HDAC) inhibitors with the image of the brain of the subject before administration, the positive control brain image comprising reactive astrocytes, and the negative control brain image comprising non-reactive or resting astrocytes.

In certain embodiments, the HDAC inhibitors comprise a pan-HDAC inhibitor (such as AR-42, Belinostat, Givinostat, Dacinostat, M344, Panobinostat, Abexinostat, Pracinostat, Quisinostat, Rocilinostat, Scriptaid, Trichostatin A, and Vorinostat).

In certain embodiments, said one or more HDAC inhibitors target HDAC1, HDAC2, HDAC3, HDAC8, or a combination thereof. In certain embodiments, said one or more HDAC inhibitors are specific for HDAC3, such as RGFP966, BRD3308, or HDAC3-IN-T247 (T247). In certain embodiments, the HDAC inhibitor specific for HDAC3 is RGFP966.

In certain embodiments, the disease or disorder is a disease or disorder of the brain, cerebral injury, brain and systemic disease, neurological disease, cerebral injury, disease associated with loss or reduction of level of calbindin, neurotoxicity, Niemann-Pick disease, Niemann-Pick Type A disease, Niemann-Pick Type B disease, Niemann-Pick Type C disease, neurodegenerative disorder, traumatic brain injury (TBI), autism, Alzheimer's, inflammatory disorder, neuroinflammatory disorder, neuroinflammation due to lysosomal storage disorder, lysosomal storage disorder, Gliobastoma multiforme, HIV, HIV associated cognitive deficits, non-neurological disease, brain tumor, disease responsive to treatment with histone deacetylase (HDAC) inhibitor, disease involving plasma concentration of vorinostat (SAHA), disease responsive to treatment with SAHA, disease where effect of SAHA is observed in animal model, encephalopathy, epilepsy, cerebrovascular disease, disease responsive to penetration of drug through the blood-brain barrier, Parkinsons, Amyotrophic Lateral Sclerosis, activator deficiency/GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, Gaucher's disease, Gaucher disease (types I-III), GM1 gangliosidosis, I-cell disease/muco lipidosis II, infantile free sialic acid storage disease/ISSD, juvenile hexosaminidase A deficiency, Krabbe disease, metachromatic leukodystrophy, mucopolysaccharidoses disorders, pseudo-Hurler polydystrophy/mucolipidosis IIIA, MPSI Hurler syndrome, MPSI Scheie syndrome, MPS I Hurler-Scheie syndrome, MPS II Hunter syndrome, Sanfilippo syndrome, Morquio syndrome, MPS FX hyaluronidase deficiency, MPS VI Maroteaux-Lamy, MPS VII Sly syndrome, mucolipidosis I/sialidosis, multiple sulfatase deficiency, neuronal ceroid lipofuscinoses, Pompe disease, Sandhoff disease, Schindler disease, Salla disease, Tay-Sachs, spinal muscular atrophy, Huntington's disease, or a combination thereof.

In certain embodiments, the disease is a neurodegenerative disease, a myelin or white matter disease, a genetic disease, an inflammatory disease or disorder, an acquired disease or disorder of the central nervous system, or a combination thereof.

In certain embodiments, the neurodegenerative disease is one or more of Alexander disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, diabetic neuropathy, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), wet or dry macular degeneration, Multiple System Atrophy, multiple sclerosis, Niemann Pick disease, Parkinson's disease, Pelizaeus-Merzbacher Disease, photoreceptor degenerative diseases such as retinitis pigmentosa and associated diseases, Pick's disease, primary lateral sclerosis, prion diseases, Progressive Supranuclear Palsy, Refsum's disease, Sandhoffs disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, or a combination thereof.

In certain embodiments, the myelin or white matter disease is acute disseminated encephalomyelitis, destructive leukoencephalopathy, leukoencephalitis, multiple sclerosis, cerebral palsy, periventricular leukomalacia (PVL), hypoxia induced white matter disease, neuromyelitis optica, adult-onset autosomal dominant leukodystrophy (ADLD), Aicardi-Goutieres syndrome, Alexander's disease, CADASIL, Canavan disease, CARASIL, cerebrotendinous xanthomatosis, childhood ataxia and cerebral hypomyelination (CACH)/vanishing white matter disease (VWMD), Fabry disease, fucosidosis, GM1 gangliosidosis, Krabbe's disease, L-2-hydroxyglutaric aciduria, megalencephalic leukoencephalopathy with subcortical cysts, metachromatic leukodystrophy, multiple sulfatase deficiency, Pelizaeus-Merzbacher disease, Pol III-Related Leukodystrophies, Refsum disease, salla disease (free sialic acid storage disease), Sjogren-Larsson syndrome, X-linked adrenoleukodystrophy, Zellweger syndrome spectrum disorders, or a combination thereof.

In certain embodiments, the genetic disease is Huntington's disease, adrenaleukodystrophy, Krabbe's disease, Pelizeaus-Merzbacher disease, vanishing white matter disease, Alexander's disease, metachromatic leukodystrophy, Megalencephalic Leukodystrophy with subcortical Cysts, Canavan's disease, lysosomal storage disorders, leukodystrophies, or a combination thereof.

In certain embodiments, the inflammatory disease or disorder is multiple sclerosis, neuromyelitis optica, chronic inflammatory demyelinating polyneuropathy, acute disseminated encephalomyelitis, acute optic neuritis, transverse myelitis, encephalitis, meningitis, or a combination thereof.

In certain embodiments, the acquired disease or disorder of the central nervous system is traumatic brain injury, chronic traumatic encephalopathy, stroke, brain metastasis, neuroaids, cancer related cognitive impairment, immune effector cell-associated neurotoxicity syndrome (ICANS), or a combination thereof. In certain embodiments, the disease or disorder is Alzheimer's disease.

Another aspect of the invention provides a method of inhibiting RelA/P65-mediated transcription in a reactive astrocyte, the method comprising: contacting the reactive astrocyte with an effective amount of one or more histone deacetylase (HDAC) inhibitors (such as HDAC3-specific inhibitor). In certain embodiments, said HDAC inhibitors inhibit nuclear translocation of RelA/P65.

Another aspect of the invention provides a method of decreasing very long chain fatty acids (VLCFA) in a reactive astrocyte, the method comprising: contacting the reactive astrocyte with an effective amount of one or more histone deacetylase (HDAC) inhibitors (such as HDAC3-specific inhibitor). In certain embodiments, said HDAC inhibitors inhibit VLCFA production.

In certain embodiments, said one or more HDAC inhibitors comprise a pan-HDAC inhibitor (such as AR-42, Belinostat, Givinostat, Dacinostat, M344, Panobinostat, Abexinostat, Pracinostat, Quisinostat, Rocilinostat, Scriptaid, Trichostatin A, and Vorinostat).

In certain embodiments, said one or more HDAC inhibitors target HDAC1, HDAC2, HDAC3, HDAC8, or a combination thereof. In certain embodiments, said one or more HDAC inhibitors are specific for HDAC3, such as RGFP966, BRD3308, or HDAC3-IN-T247 (T247). In certain embodiments, the HDAC inhibitor specific for HDAC3 is RGFP966.

In certain embodiments, the reactive astrocyte is in a subject suspected of having, or at a high risk of having, a disease or disorder associated with reactive astrocytes, and/or has been identified as having reactive astrocytes.

Another aspect of the invention provides a method of promoting CNS tissue repair in a subject in need thereof, the method comprising: administering to the subject a therapeutically effective amount of one or more histone deacetylase (HDAC) inhibitors, wherein the one or more HDAC inhibitors suppress reactive astrocytes (e.g., suppress the formation, maintenance, and/or function of the reactive astrocytes, such as GBP2⁺ reactive astrocytes).

In certain embodiments, said CNS tissue has neuronal demyelination, and/or axonal damage, and wherein said CNS tissue repair comprises axonal remyelination.

In certain embodiments, said one or more HDAC inhibitors comprise a pan-HDAC inhibitor (such as AR-42, Belinostat, Givinostat, Dacinostat, M344, Panobinostat, Abexinostat, Pracinostat, Quisinostat, Rocilinostat, Scriptaid, Trichostatin A, and Vorinostat).

In certain embodiments, said one or more HDAC inhibitors target HDAC1, HDAC2, HDAC3, HDAC8, or a combination thereof. In certain embodiments, said one or more HDAC inhibitors are specific for HDAC3, such as RGFP966, BRD3308, or HDAC3-IN-T247 (T247). In certain embodiments, the HDAC inhibitor specific for HDAC3 is RGFP966.

It should be understood that any one embodiment described herein, including those only described in the examples or claims, can be combined with any one or more embodiments of the invention unless expressly disclaimed or being improper.

### BRIEF DESCRIPTION OF THE DRAWINGS

FIG. 1 shows ranked efficiency of specific HDAC inhibition based on published results, viability of astrocytes treated with compound, and the percent of proinflammatory toxic astrocytes when treated with 2 μM of each compound that was in the primary screen. Individual HDAC inhibitors screen in the primary high-throughput screen have varied efficacy against HDAC isotypes with the exception of RGFP966, which is specific for HDAC3. All HDAC inhibitors are well tolerated and are not toxic to astrocytes. All HDAC inhibitors including the HDAC3-specific inhibitor RGFP966 robustly inhibit the formation of proinflammatory toxic astrocytes.

FIG. 2 shows dose curve responses of the various HDAC inhibitors. Data shows the ability of validated HDAC inhibitors to block the formation of damaging reactive astrocytes and multiple concentrations.

FIG. 3 shows that HDAC inhibitors block the functional acquisition of antigen cross presentation by reactive astrocytes. Reactive astrocytes acquire the ability to cross present antigen on MHC Class I. Treatment with HDAC inhibitors RGFP966 and HDAC3-IN-T247 (T247) both decrease the ability of reactive astrocytes to cross present antigen on MHC Class I.

FIG. 4 shows that HDAC inhibitors block the secretion of the cytokine CCL5 by reactive astrocytes. Reactive astrocytes secrete cytokines including CCL5. Treatment with the HDAC inhibitors RGFP966 and T247 significantly decrease the secretion of CCL5 by reactive astrocytes.

FIG. 5 shows that HDAC inhibition blocks the formation of damaging reactive astrocytes in vivo. Mice pretreated with the brain penetrating HDAC inhibitor RGFP966 do not form reactive GBP2⁺ astrocytes in response to systemic lipopolysaccharide injections.

FIG. 6 shows that HDAC3 inhibition by RGFP966 treatment decreased expression of genes associate with damaging reactive astrocytes (DRA), while increasing expression of genes associated with beneficial reactive astrocytes (BRA) in mice.

FIG. 7 shows that HDAC3 inhibition by RGFP966 treatment decreased expression of genes associated with DRAs, Gbp2 (Guanylate Binding Protein 2) and Psmb8 (Proteasome 20S Subunit Beta 8) in cultured human astrocytes.

FIG. 8 shows that HDAC3 inhibition by RGFP966 and T247 decreases nuclear RelA/P65.

FIG. 9 shows that HDAC inhibitors blocked RelA/P65 transcriptional activity.

FIG. 10 shows tissue repair in vehicle-treated LPC mice. Animals that were injected daily with 10 mg/kg RGFP966 beginning at −1 dpl showed accelerated tissue repair at 12 dpl. Total remyelinated, percentage of axons remyelinated, total axons in lesions, and axons/mm² increased with RGFP966 treatment in the lesion of LPC injected mice.

FIG. 11 shows that genetic knockout of HDAC3 is sufficient to block the formation of GBP2⁺ reactive astrocytes.

FIG. 12 shows that treatment with the HDAC inhibitor RGFP966 can decrease the levels of toxic very long chain fatty acids (VLCFA) produced by reactive astrocytes.

FIG. 13 shows HDAC3 inhibition with RGFP966 protects retinal ganglion cells (RGCs) from neurodegeneration following optic nerve crush. The optic nerve of adult mice was surgically accessed and crushed. Followed by the crush injury, mice were injected daily with either vehicle or 10 mg/kg RGFP966 daily via i.p. for 14 days. 14 days after optic nerve crush surgery, retina were harvested and stained for the RGC marker BRN3A, and the pan neuronal maker BIII-tubulin.

### DETAILED DESCRIPTION OF THE INVENTION

### 1. Overview

The invention described herein provides treatments for various neurodegenerative diseases (NDs) associated with astrocytes that have polarized to reactive states, by using HDAC3-specific inhibitors.

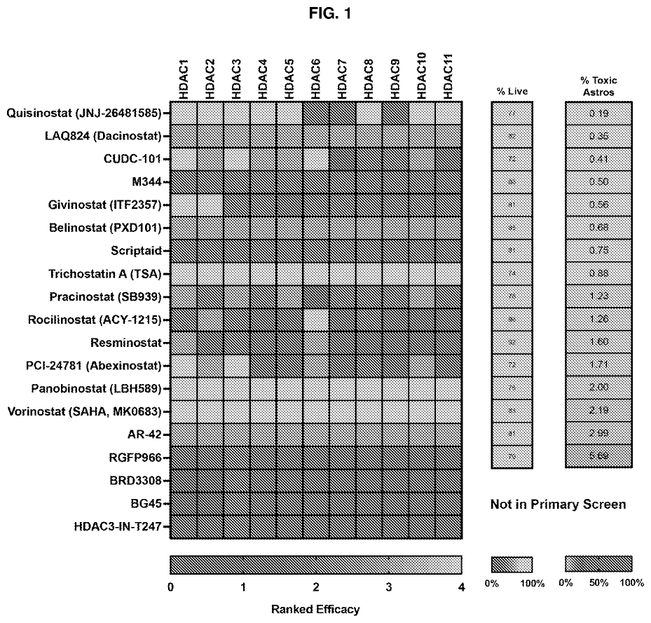

The invention is partly based on the discovery that, based on a high-throughput drug screen performed in resting astrocytes, HDAC inhibitors, especially HDAC3-specific inhibitors, inhibit the formation of reactive astrocytes. Specifically, fourteen (14) HDAC inhibitors were validated in secondary assays (Table 1), including pan-HDAC inhibitors and Class I HDAC inhibitors. Notably, no Class II or Class III/Sirtuin inhibitors were effective in this screen, suggesting that the effective target for the HDAC inhibitors are Class I HDACs (HDAC8 1/2/3/8) (FIG. 1).

Data also showed that, during a reactive state change, astrocytes undergo significant chromatin reorganization driven in part by NF-κB that directly targets and increases expression of genes responsible for reactive astrocyte functions. It was further demonstrated that HDAC3 is required for such reactive astrocyte state change, and that inhibiting HDAC3 in vivo leads to improved axonal health and increased repair following CNS injury.

Collectively, these results provided greater mechanistic understanding of reactive astrocyte state change, and identified Class I HDAC8 as a reactive astrocyte-targeted therapeutic for treating various neurodegenerative diseases associated with damaging reactive astrocytes.

### 2. Treatable Reactive Astrocyte-Mediated Diseases

One aspect of the invention provides a method for treating a subject with a disease or disorder associated with reactive astrocytes, comprising administering to the subject a therapeutically effective amount of one or more histone deacetylase (HDAC) inhibitors, wherein the one or more HDAC inhibitors suppress reactive astrocytes (e.g., suppress the formation, maintenance, and/or function of the reactive astrocytes).

In a related aspect, the invention provides a method of decreasing, in an subject in need thereof, reactive astrocyte induction/formation/conversion from resting astrocytes, the method comprising: administering to the subject an effective amount of one or more histone deacetylase (HDAC) inhibitors (such as HDAC3-specific inhibitor) to suppress reactive astrocytes; wherein the subject optionally is suspected of having, or at a high risk of having, a disease or disorder associated with reactive astrocytes, and/or has been identified as having reactive astrocytes.

In yet another related aspect, the invention provides a method of inhibiting RelA/P65-mediated transcription in a reactive astrocyte, the method comprising: contacting the reactive astrocyte with an effective amount of one or more histone deacetylase (HDAC) inhibitors (such as HDAC3-specific inhibitor).

In still another aspect, the invention provides a method of promoting CNS tissue repair in a subject in need thereof, the method comprising: administering to the subject a therapeutically effective amount of one or more histone deacetylase (HDAC) inhibitors, wherein the one or more HDAC inhibitors suppress reactive astrocytes (e.g., suppress the formation, maintenance, and/or function of the reactive astrocytes, such as GBP2⁺ reactive astrocytes).

In certain embodiments, the one or more HDAC inhibitors comprise a pan-HDAC inhibitor (such as AR-42, Belinostat, Givinostat, Dacinostat, M344, Panobinostat, Abexinostat, Pracinostat, Quisinostat, Rocilinostat, Scriptaid, Trichostatin A, and Vorinostat).

In certain embodiments, the one or more HDAC inhibitors target HDAC1, HDAC2, HDAC3, HDAC8, or a combination thereof.

In certain embodiments, the one or more HDAC inhibitors are specific for HDAC3, such as RGFP966, BRD3308, or HDAC3-IN-T247 (T247). In certain embodiments, the HDAC inhibitor specific for HDAC3 is RGFP966.

Normal astrocytes are stellate cells in the central nervous system that perform a myriad of tasks that are necessary for the proper function of the brain. Astrocytes, however, are also involved in the neuroinflammatory response. Neuroinflammation refers to a state of reactivity of astrocytes and microglia induced by various pathological conditions, and may be associated with the recruitment of peripheral macrophages and lymphocytes. Reactive astrocytes and microglia mediate the innate immune responses in the brain. Astrocytes become reactive in response to virtually all pathological situations in the brain, both following acute injuries (stroke, trauma, axotomy, ischemia, infection, inflammation, traumatic brain injury), and during progressive diseases such as tumors, epilepsy, and neurodegenerative diseases (ND).

Here, astrocyte reactivity or reactive astrocytes refer to astrocytes that respond to any pathological condition in the CNS. Astrocytes are considered reactive when they become hypertrophic and overexpress the intermediate filament GFAP (glial fibrillary acidic protein)-two of the most universal hallmarks of reactivity. But this definition does not exclude many additional transcriptional, morphological and functional changes that occur in a disease-specific manner. In general, astrocyte reactivity involves the activation of transcriptional program(s) triggered by specific signaling cascades that results in long-lasting changes in morphology and function, persisting over several hours, days or even decades.

Astrocyte reactivity is not unique to human. It has been observed in many mammalian and bird species. Even in lampreys, newts and frogs, astrocyte-like cells react to injury and form a glial bridge promoting axonal regeneration. In Drosophila, glial cells with some typical astrocyte functions display strong phagocytic activity and morphological changes following neuronal degeneration.

Astrocyte reactivity was originally characterized by morphological changes-hypertrophy (enlarged cell body and processes), remodeling of processes, etc. It is also characterized by transcriptional and functional changes such as the overexpression of GFAP. However, it was unclear how the normal supportive functions of astrocytes are altered by their reactive state. In neurodegenerative diseases, for example, neuronal dysfunction and astrocyte reactivity take place over several years or even decades, making the issue even more complex and highly debated, with several conflicting reports published recently.

Regardless, research has shown that astrocyte reactivity is a shared and central feature in numerous neurodegenerative diseases, including Multiple Sclerosis (MS), Alzheimer's Disease (AD), Huntington's diseases (HD), Amyotrophic Lateral Sclerosis (ALS), and Parkinson's Disease (PD).

Specifically, astrocyte reactivity can be detected in the brain of AD patients with imaging and proteomic techniques even before the onset of symptoms. Foci of reactive astrocytes are also detected at early stages in some mouse models, even before amyloid deposition. Reactive astrocytes are usually found around amyloid plaques. Patches of reactive astrocytes may also be found in the absence of plaques in patients. In addition, atrophied astrocytes may be located at a distance from plaques in some mouse models.

Astrocyte reactivity is also an early feature of HD —GFAP immunoreactivity is detected in the striatum of presymptomatic carriers, and it increases with disease progression. Strikingly, no clear evidence of astrocyte reactivity exists in most HD models. Instead, HD astrocytes show functional alterations in the absence of the main features of reactivity-hypertrophy and high GFAP expression.

Reactive astrocytes are observed in both ALS patients and ALS models. They appear in vulnerable regions, and the degree of reactivity correlates with the level of neurodegeneration.

In PD, though the involvement of microglial cells in PD has been more extensively studied than that of astrocytes, astrocyte reactivity is detected in the SNpc (substantia nigra pars compacta) of patients with PD, individuals intoxicated with MPTP (1-methyl-4-phenyl-1,2,3,6-tetrahydropyridine) and in animal models.

Thus the methods of the invention can be used to treat any neurodegenerative diseases or disorders associated with reactive astrocytes, or to prevent or retard the onset, progression, or worsening of at least a symptom of such neurodegenerative diseases or disorders, at least partly by inhibiting the formation of reactive astrocytes.

In certain embodiments, the treatable neurodegenerative disease or disorder is one or more of disease or disorder of the brain, cerebral injury, brain and systemic disease, neurological disease, cerebral injury, disease associated with loss or reduction of level of calbindin, neurotoxicity, Niemann-Pick disease, Niemann-Pick Type A disease, Niemann-Pick Type B disease, Niemann-Pick Type C disease, neurodegenerative disorder, traumatic brain injury (TBI), autism, Alzheimer's, inflammatory disorder, neuroinflammatory disorder, neuroinflammation due to lysosomal storage disorder, lysosomal storage disorder, Gliobastoma multiforme, HIV, HIV associated cognitive deficits, brain tumor, disease responsive to treatment with histone deacetylase (HDAC) inhibitor, disease involving plasma concentration of vorinostat (suberoylanilide hydroxamic acid (SAHA)), disease responsive to treatment with SAHA, disease where effect of SAHA is observed in animal model, encephalopathy, epilepsy, cerebrovascular disease, disease responsive to penetration of drug through the blood-brain barrier, Parkinson's disease, Amyotrophic Lateral Sclerosis, activator deficiency/GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, Gaucher's disease, Gaucher disease (types I-III), GM1 gangliosidosis, I-cell disease/muco lipidosis II, infantile free sialic acid storage disease/ISSD, juvenile hexosaminidase A deficiency, Krabbe disease, metachromatic leukodystrophy, mucopolysaccharidoses disorders, pseudo-Hurler polydystrophy/mucolipidosis IIIA, MPSI Hurler syndrome, MPSI Scheie syndrome, MPS I Hurler-Scheie syndrome, MPS II Hunter syndrome, Sanfilippo syndrome, Morquio syndrome, MPS FX hyaluronidase deficiency, MPS VI Maroteaux-Lamy, MPS VII Sly syndrome, mucolipidosis I/sialidosis, multiple sulfatase deficiency, neuronal ceroid lipofuscinoses, Pompe disease, Sandhoff disease, Schindler disease, Salla disease, Tay-Sachs spinal muscular atrophy, Huntington's disease, or a combination thereof.

In certain embodiments, the disease is selected from the group consisting of a neurodegenerative disease, a myelin or white matter disease, a genetic disease, an inflammatory disease or disorder, an acquired disease or disorder of the central nervous system, and a combination thereof.

In certain embodiments, the neurodegenerative disease is Alexander disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, diabetic neuropathy, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), wet or dry macular degeneration, Multiple System Atrophy, multiple sclerosis, Niemann Pick disease, Parkinson's disease, Pelizaeus-Merzbacher Disease, photoreceptor degenerative diseases such as retinitis pigmentosa and associated diseases, Pick's disease, primary lateral sclerosis, prion diseases, Progressive Supranuclear Palsy, Refsum's disease, Sandhoffs disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, or a combination thereof.

In certain embodiments, the myelin or white matter disease is acute disseminated encephalomyelitis, destructive leukoencephalopathy, leukoencephalitis, multiple sclerosis, cerebral palsy, periventricular leukomalacia (PVL), hypoxia induced white matter disease, neuromyelitis optica, adult-onset autosomal dominant leukodystrophy (ADLD), Aicardi-Goutieres syndrome, Alexander's disease, CADASIL, Canavan disease, CARASIL, cerebrotendinous xanthomatosis, childhood ataxia and cerebral hypomyelination (CACH)/vanishing white matter disease (VWMD), Fabry disease, fucosidosis, GM1 gangliosidosis, Krabbe's disease, L-2-hydroxyglutaric aciduria, megalencephalic leukoencephalopathy with subcortical cysts, metachromatic leukodystrophy, multiple sulfatase deficiency, Pelizaeus-Merzbacher disease, Pol III-Related Leukodystrophies, Refsum disease, salla disease (free sialic acid storage disease), Sjogren-Larsson syndrome, X-linked adrenoleukodystrophy, Zellweger syndrome spectrum disorders, or a combination thereof.

In certain embodiments, the neurodegenerative disease is Alexander disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, diabetic neuropathy, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), wet or dry macular degeneration, Multiple System Atrophy, multiple sclerosis, Niemann Pick disease, Parkinson's disease, Pelizaeus-Merzbacher Disease, photoreceptor degenerative diseases such as retinitis pigmentosa and associated diseases, Pick's disease, primary lateral sclerosis, prion diseases, Progressive Supranuclear Palsy, Refsum's disease, Sandhoffs disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, or a combination thereof.

In certain embodiments, the myelin or white matter disease is acute disseminated encephalomyelitis, destructive leukoencephalopathy, leukoencephalitis, multiple sclerosis, cerebral palsy, periventricular leukomalacia (PVL), hypoxia induced white matter disease, neuromyelitis optica, adult-onset autosomal dominant leukodystrophy (ADLD), Aicardi-Goutieres syndrome, Alexander's disease, CADASIL, Canavan disease, CARASIL, cerebrotendinous xanthomatosis, childhood ataxia and cerebral hypomyelination (CACH)/vanishing white matter disease (VWMD), Fabry disease, fucosidosis, GM1 gangliosidosis, Krabbe's disease, L-2-hydroxyglutaric aciduria, megalencephalic leukoencephalopathy with subcortical cysts, metachromatic leukodystrophy, multiple sulfatase deficiency, Pelizaeus-Merzbacher disease, Pol III-Related Leukodystrophies, Refsum disease, salla disease (free sialic acid storage disease), Sjogren-Larsson syndrome, X-linked adrenoleukodystrophy, Zellweger syndrome spectrum disorders, or a combination thereof.

In certain embodiments, the genetic disease is one or more of Huntington's disease, adrenaleukodystrophy, Krabbe's disease, Pelizeaus-Merzbacher disease, vanishing white matter disease, Alexander's disease, metachromatic leukodystrophy, Megalencephalic Leukodystrophy with subcortical Cysts, Canavan's disease, lysosomal storage disorders, leukodystrophies, or a combination thereof.

In certain embodiments, the inflammatory disease or disorder is multiple sclerosis, neuromyelitis optica, chronic inflammatory demyelinating polyneuropathy, acute disseminated encephalomyelitis, acute optic neuritis, transverse myelitis, encephalitis, meningitis, or a combination thereof.

In certain embodiments, the acquired disease or disorder of the central nervous system is traumatic brain injury, chronic traumatic encephalopathy, stroke, brain metastasis, HIV associated cognitive impairment, neuroaids, cancer related cognitive impairment, immune effector cell-associated neurotoxicity syndrome (ICANS), or a combination thereof.

In certain embodiments, the disease or disorder is Alzheimer's disease, Parkinson's disease, multiple sclerosis, Huntington's disease, or amyotrophic lateral sclerosis.

In certain embodiments, the disease or disorder is Alzheimer's disease.

### 3. HDAC3 Inhibitors

Numerous HDAC3 inhibitors (specific or non-specific inhibitors) are known in the art, and can be used in the methods of the invention.

In certain embodiments, the HDAC3 inhibitor comprises Abexinostat (PCI-24781), Apicidin (OSI2040), AR-42, Belinostat (PXD101), BG45, BML-210, BML-281, BMN290, BRD0302, BRD2283, BRD3227, BRD3308, BRD3349, BRD3386, BRD3493, BRD4161, BRD4884, BRD6688, BRD8951, BRD9757, BRD9757, CBHA, Chromopeptide A, Citarinostat (ACY-214), CM-414, compound 25, CRA-026440, Crebinostat, CUDC-101, CUDC-907, Curcumin, Dacinostat (LAQ824), Depudecin, Domatinostat (4SC-202), Droxinostat, Entinostat (MS0275), EVX001688, FR901228, FRM-0334, Givinostat, HDAC1-4b, HDAC1-109, HPOB, 12, KD5170, LB-205, M344, Martinostat, Merck60 (BRD6929), Mocetinostat (MGCD0103), OBP-801, Oxamflatin, Panobinostat (LBH589), PCI-34051, PCI-48000, Pracinostat (SB939), Pyroxamide, Quisinostat (JNJ-26481585), Resminostat, RG2833 (RGFP109), RGFP963, RGFP966, RGFP968, Rocilinostat (ACY-1215), Romidepsin (FK228), Scriptaid, sodium phenylbutyrate, Splitomicin, T247, Tacedinaline (CI994), Trapoxin, Trichostatin A (TSA), Tucidinostat (chidamide), Valproic acid, vorinostat (SAHA), W2, MC1742, MC2625, A8B4, A14B3, A12B4, A14B4, A7B4, CI-994, or a combination thereof.

In certain embodiments, the HDAC3 inhibitor comprises HDACi 4b, Entinostat (MS-275), BG45, RG2833 (RGFP109), or RGFP966.

In certain embodiments, the HDAC3 inhibitor comprises sodium butyrate, phenylacetate, phenylbutyrate, valproic acid, tributyrinpivaloyloxymethyl butyrate, pivanex®, trichostatinA (TSA), trichostatin C, trapoxins A and B, depudecin, cyclic hydroxamic-acid containing peptide (CHAPs), apicidin or OSI-2040, suberoylanilide hydroxamic acid (SAHA), oxamflatindepsipeptide, FK228, scriptaid, biarylhydroxamate inhibitor, A-161906, JNJ16241199, PDX 101, MS-275, or CI-994, or a combination thereof.

In certain embodiments, the HDAC3-specific inhibitor comprises a compound of Formula (I) or a pharmaceutically acceptable salt, hydrate, solvate, or prodrug thereof, as described in WO2020252323A1 (incorporated herein by reference),

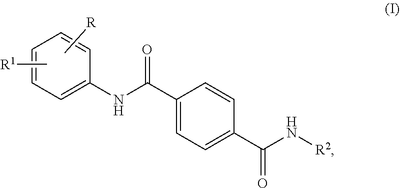

wherein:
R is selected from the group consisting of fluoro, bromo, chloro, —NH₂, —OH, —SH, —NHR₃, —N(R₃)₂, OR₃, SR₃, NO₂, thienyl, and CN;
R1 is selected from the group consisting of fluoro, bromo, chloro, —NH₂, —OH, —SH, —NHR₃, —N(R₃)₂, OR₃, SR₃, NO₂, thienyl, and CN;
R2 is selected from the group consisting of C₅-C₁₀ aryl, C₅-C₁3 heteroaryl, C₃-C₁₀ cycloalkyl, C₃-C₁₀ heterocycloalkyl, C₁-C₈ alkyl-C₅-C₁₀ aryl, C₁-C₈ alkyl-C₅-Cₙ heteroaryl, C₁-C₆ alkyl-C₃-C₁₀ cycloalkyl, C₁-C₆ alkyl-C₃-C₁₀ heterocycloalkyl, and —linker-biotin;
wherein C₅-C₁₀ heteroaryl, C₅-C₁₀ aryl, and C₁-C₈ alkyl are optionally substituted with one to three halo, phenyl, —C(O)Me, —OMe, methyl, NO₂, —SO₂Me, C₈ heterocycloalkyl, C₅-C₈heteroaryl, and CF₃; and
R3 is independently, at each occurrence, selected from the group consisting of H, C₁-C₈ alkyl, and C₁-C₈ alkoxy.

In certain embodiments, the HDAC3-specific inhibitor comprises a compound of Formula (I) or a pharmaceutically acceptable salt, hydrate, solvate, or prodrug thereof, as described in WO2019140322A1 (incorporated herein by reference),

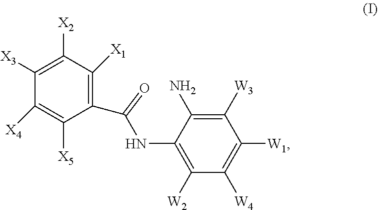

wherein:
W₁, W₂, W₃, and W₄ are each independently selected from hydrogen, fluorine, chlorine, bromine, CF₃, CH₃, and deuterium, provided that at least one of W₁, W₂, W₃, or W₄ is not hydrogen;
X₁ and X₅ are each independently selected from hydrogen, halogen and C₁-C₃ alkyl;
X₂, X₃, and X₄ are each independently selected from hydrogen, halogen, OR₅, C(O)R₆, OS(O)ₚR₇, NR₃R₄, NR₁C(O)R₂, NR₁S(O)ₚR₇, S(O)_{q}R₁₀, C(O)OR₁₁, C(O)NR₁₂R₁₃, OC(O)OR₁₄, OC(O)NR₁₅R₁₆, NR₁₇C(O)OR₁₅, NR₁₉C(O)NR₂₀R₂₁, C₁-C₈ alkyl, C₂-C₈ alkenyl, C₂-C₈ alkynyl, C₃-C₈ cycloalkyl, C₄-C₅ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring, and heterocyclic ring is unsubstituted or substituted with one or more Rₐ and one or two of X₂, X₃, and X₄ is hydrogen;
Rₐ is selected from halogen, OR₂₅, C₁-C₈ alkyl, CF₃, CHF₂, CH₂F, NR₂₆C(O)R₂₇, and NR₂₈R₂₉; or
X₂ and X₃ or X₄ and X₃ taken together with the atoms to which they are attached form ring selected from a C₃-C₈ cycloalkyl, C₄-C₅ cycloalkenyl, aromatic ring, 3-8 membered heteroaromatic ring and 3-8 membered saturated or partially saturated heterocyclic ring, wherein said ring is unsubstituted or substituted with one or more Rv, Rv is selected from halogen, OR₂₅, C₁-C₈ alkyl, CF₃, CHF₂, CH₂F, NR₂₆C(O)R₂₇, NR₂₈R₂₉, S(O)qR₇, S(O)_{q}R₁₀, C(O)OR₁₁, C(O)NR₁₂R₁₃, OC(O)OR₁₄, OC(O)NR₁₅R₁₆, NR₁₇C(O)OR₁₅, and NR₁₉C(O)NR₂₀R₂₁;
R₁ and R₂₆ are each independently selected from hydrogen and C₁-C₈ alkyl; R₂ is selected from hydrogen, C₁-C₈ alkyl, C₂-C₈ alkenyl, C₃-C₈ alkynyl, C₃-C₈ cycloalkyl, C₄-C₅ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring, and heterocyclic ring are unsubstituted or substituted with one or more Rb;
R₂₇ is selected from hydrogen, C₁-C₈ alkyl, C₃-C₈ alkynyl, C₃-C₈ cycloalkyl, C₄-C₅ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring, and heterocyclic ring are unsubstituted or substituted with one or more Rb;
R_{b} is selected from halogen, C₁-C₈ alkyl, CF₃, CHF₂, CH₂F, OR₂₅, NH₂, NHCH₃, N(CH₃)₂, C₃-C₈ cycloalkyl, C₄-C₅ cycloalkenyl, aromatic ring, 3-8 membered heteroaromatic ring, 3-8 membered saturated or partially saturated heterocyclic ring, wherein said cycloalkyl, cycloalkenyl, aromatic ring,
heteroaromatic ring, and heterocyclic ring is unsubstituted or substituted with one or more R_{b1}; R_{b1} is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R₃ and R₄ are each independently selected from hydrogen, C₁-C₈ alkyl, C₂-C₈ alkenyl, C₃-C₈ alkynyl, C₃-C₈ cycloalkyl, C₄-C₅ cycloakenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubstituted or substituted with one or more R_{g};
R₂₈ and R₂₉ are each independently selected from hydrogen, C₁-C₈ alkyl, C₂-C₈ alkenyl, C₃-C₈ alkynyl, C₃-C₈ cycloalkyl, C₄-C₅ cycloakenyl, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubstituted or substituted with one or more R_{g};
R_{g} is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₅ cycloalkenyl, aromatic ring, 3-8 membered heteroaromatic ring, and 3-8 membered saturated or partially saturated heterocyclic ring, wherein said cycloalkyl, cycloalkenyl, aromatic ring, heteroaromatic ring, and heterocyclic ring is unsubstituted or substituted with one or more Rₕ;
Rₕ is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R₅ and R₂₅ are each independently selected from hydrogen, C₁-C₈ alkyl, CF₃, CHF₂, CH₂F, C₂-C₈ alkenyl, C₃-C₈ alkynyl, C₃-C₈ cycloalkyl, C₄-C₈ cycloalkenyl, aromatic ring, 3-8 membered heteroaromatic ring, 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubstituted or substituted with one or more R_{c};
R_{c} is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more R_{d};
R_{d} is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R₆ is selected from hydrogen, OR₂₅, C₁-C₈ alkyl, CF₃, CHF₂, CH₂F, C₂-C₈ alkenyl, C₃-C₈ alkynyl, C₃-C₈ cycloalkyl, C₄-C₈ cycloalkenyl, aromatic ring, 3-8 membered heteroaromatic ring, and 3-8 membered saturated or partially saturated heterocyclic ring; wherein said alkyl, alkenyl, alkynyl, cycloalkyl, cycloalkenyl, aromatic ring, heteroaromatic ring and heterocyclic ring are unsubstituted or substituted with one or more Rₑ;
Rₑ is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more R_{f};
R_{f} is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R₇ is selected from C₁-C₈ alkyl, C₃-C₈ cycloalkyl, C₄-C₈ cycloalkenyl, aromatic ring, 3-8 membered heteroaromatic ring, and 3-8 membered saturated or partially saturated heterocyclic ring; wherein said alkyl, cycloalkyl, cycloalkenyl, aromatic ring, heteroaromatic ring, and heterocyclic ring are unsubstituted or substituted with one or more Rᵢ;
Rᵢ is selected from halogen, C₁-C₃ alkyl, CF₃, CHF₂, CH₂F, OH, OCH₃, NH₂, NHCH₃, and N(CH₃)₂;
R₁₀ are each independently selected from C₁-C₈ alkyl, C₃-C₈ cycloalkyl, C₄-C₅ cycloalkenyl, aromatic ring, 3-8 membered heteroaromatic ring, and 3-8 membered saturated or partially saturated heterocyclic ring; wherein said alkyl, cycloalkyl, cycloalkenyl, aromatic ring, heteroaromatic ring, and heterocyclic ring are unsubstituted or substituted with one or more Rⱼ;
Rⱼ is selected from halogen, C₁-C₃ alkyl, CF₃, CHF₂, CH₂F, OH, OCH₃, NH₂, NHCH₃, and N(CH₃)₂;
R₁₁ is selected from hydrogen, C₁-C₈ alkyl, C₂-C₈ alkenyl, C₂-C₈ alkynyl, C₃-C₈ cycloalkyl, C₄-C₅ cycloalkenyl, aromatic ring, 3-8 membered heteroaromatic ring, and 3-8 membered saturated or partially saturated heterocyclic ring; wherein said alkyl, alkenyl, alkynyl, cycloalkyl, cycloalkenyl, aromatic ring, heteroaromatic ring and heterocyclic ring are unsubstituted or substituted with one or more Rₖ;
Rₖ is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₅ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more Rₖ₁;
Rₖ₁ is selected from halogen, C₁-C₃ alkyl, CF₃, CHF₂, CH₂F, OH, OCH₃, SO₂CH₃, NH₂, NHCH₃, and N(CH₃)₂;
R₁₂ and R₁₃ are each independently selected from hydrogen, C₁-C₈ alkyl, C₂-C₈ alkenyl, C₃-C₈ alkynyl, C₃-C₈ cycloalkyl, C₄-C₅ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubstituted or substituted with one or more R1; R1 is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₅ cycloalkenyl, aromatic ring, 3-8 membered heteroaromatic ring, and 3-8 membered saturated or partially saturated heterocyclic ring, wherein said cycloalkyl, cycloalkenyl, aromatic ring, heteroaromatic ring, and heterocyclic ring is unsubstituted or substituted with one or more Rₘ;
Rₘ is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R₁₄ is selected from C₁-C₈ alkyl, C₂-C₈ alkenyl, C₂-C₈ alkynyl, C₃-C₈ cycloalkyl, C₄-C₈ cycloalkenyl, and aromatic ring; wherein said alkyl, alkenyl, alkynyl, cycloalkyl, cycloalkenyl, and aromatic ring are unsubstituted or substituted with one or more Rₙ;
Rₙ is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more Rₙ₁;
Rₙ₁ is selected from halogen, C₁-C₃ alkyl, CF₃, CHF₂, CH₂F, OH, OCH₃, SO₂CH₃, NH₂, NHCH₃, and N(CH₃)₂; R₁₅ and R₁₆ are each independently selected from hydrogen, C₁-C₈ alkyl, C₂-C₈ alkenyl, C₃-C₈ alkynyl, C₃-C₈ cycloalkyl, C₄-C₈ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubstituted or substituted with one or more Rₒ;
Rₒ is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, and 3-8 membered saturated or partially saturated heterocyclic ring, wherein said cycloalkyl, cycloalkenyl, aromatic ring, heteroaromatic ring, and heterocyclic ring is unsubstituted or substituted with one or more Rₚ;
Rₚ is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R₁₇ and R₁₉ are each independently selected from hydrogen and C₁-C₈ alkyl;
R₁₈ is selected from C₁-C₈ alkyl, C₂-C₈ alkenyl, C₂-C₈ alkynyl, C₃-C₈ cycloalkyl, C₄-C₈cycloalkenyl, aromatic ring, 3-8 membered heteroaromatic ring, and 3-8 membered saturated heterocyclic ring; wherein said alkyl, alkenyl, alkynyl, cycloalkyl, cycloalkenyl, heteroaromatic ring, heterocyclic ring, and aromatic ring are unsubstituted or substituted with one or more R_{q};
R_{q} is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more R_{q1}; R_{q1} is selected from halogen, C₁-C₃ alkyl, CF₃, CHF₂, CH₂F, OH, OCH₃, SO₂CH₃, NH₂, NHCH₃, and N(CH₃)₂;
R₂₀ and R₂₁ are each independently selected from selected from hydrogen, C₁-C₈ alkyl, C₂-C₈ alkenyl, C₃-C₈ alkynyl, C₃-C₈ cycloalkyl, C₄-C₅ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubstituted or substituted with one or more Rᵣ;
Rᵣ is selected from halogen, C₁-C₃ alkyl, CF₃, CHCF₂, CH₂F, OH, OCH₃, NH₂, NHCH₃, N(CH₃)₂, C₃-C₈ cycloalkyl, C₄-C₅ cycloalkenyl, aromatic ring, 3-8 membered heteroaromatic ring, and 3-8 membered saturated or partially saturated heterocyclic ring, wherein said cycloalkyl, cycloalkenyl, aromatic ring, heteroaromatic ring, and heterocyclic ring is unsubstituted or substituted with one or more R₈;
R₈ is selected from halogen, C₁-C₃ alkyl, CF₃, CHF₂, CH₂F, OH, OCH₃, SOCH₃, NH₂, SO₂CH₃, NHCH₃, and N(CH₃)₂; and p and q are each independently selected from 0, 1, and 2.

In certain embodiments, the HDAC3-specific inhibitor comprises a compound of Formula (I) or a pharmaceutically acceptable salt, hydrate, solvate, or prodrug thereof, as described in WO2018223122A1 (incorporated herein by reference),

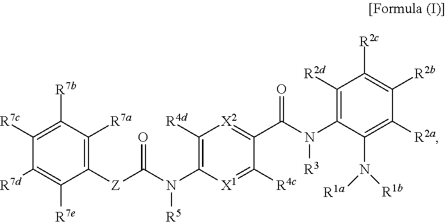

wherein:
R₁ₐ and R_{1b} are independently selected from hydrogen, C₁₋₈ alkyl, C₃₋₈ cycloalkyl, C₂₋₈alkenyl, and C₂₋₈ alkynyl;
R₂ₐ is selected from —R^{c}, —OR^{c}, —N(R^{c})₂, —SR^{c}, —SO₂R^{c}, —SO₂N(R^{c})₂; —C(O)R^{c}, OC(O)R^{C}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R^{2b} is selected from —R^{c}, —OR^{c}, —N(R^{c})₂, —SR^{c}, —SO₂R^{c}, —SO₂N(R^{c})₂; —C(O)R^{c}, —OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R^{2c} is selected from —R^{c}, —OR^{c}, —N(R^{c})₂, —SR^{c}, —SO₂R_{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R^{2d} is each independently selected from —R^{c}, —OR^{c}, —N(R^{c})₂, —SR^{c}, —SO₂R_{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R³ is R_{c};
X¹ is selected from CR^{4b} and N;
X² is selected from CR^{4a} and N;
when present, R^{4a} is selected from —R^{c}, —OR^{c}, —N(R^{c})₂, —SR^{c}, —SO₂R_{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
when present, R^{4b} is selected from —R^{c}, —OR^{c}, —N(R^{c})₂, —SR^{c}, —SO₂R_{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂; R^{4c} is selected from —R^{c}, —OR^{c}, —N(R^{c})₂, —SR^{c}, —SO₂R_{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R^{4d} is selected from—R_{c}, —OR^{c}, —N(R_{c})₂, —SR^{c}, —SO₂R_{c}, —SO₂N(R_{c})₂; —C(O)R_{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R⁵ is R_{c};

Z has the formula:

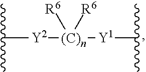

wherein Y¹ and Y² are independently selected from O, S, NR^{c}, or a chemical bond;
n is an integer selected from 0, 1, 2, 3 and 4;
R⁶ is in each case independently selected from —R^{c}, —OR^{c}, —N(R^{c})₂, —SR^{c}, —SO₂R_{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
wherein any two or more R⁶ groups may together form a ring, any adjacent R⁶ groups may together form a double bond or triple bond, and any two germinal R⁶ groups may together form an olefin, carbonyl, or imine; and
R^{7a} is selected from —R^{c}, —OR^{c}, —N(R^{c})₂, —SR^{c}, —SO₂R^{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R^{7b} is selected from —R^{c}, —OR^{c}, —N(R^{c})₂, —SR^{c}, —SO₂R^{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R^{7c}, is selected from —R^{c}, —OR^{c}, —N(R^{c})₂, —SR^{c}, —SO₂R^{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R^{7d} is selected from —R^{c}, —OR^{c}, —N(R^{c})₂, —SR^{c}, —SO₂R^{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂;
R^{7c} is selected from —R^{c}, —OR^{c}, —N(R^{c})₂, —SR^{c}, —SO₂R^{c}, —SO₂N(R^{c})₂; —C(O)R^{c}, OC(O)R^{c}, —COOR^{c}, —C(O)N(R^{c})₂, —OC(O)N(R^{c})₂, —N(R^{c})C(O), —N(R^{c})C(O)N(R^{c})₂, —F, —Cl, —Br, —I, —CN, —NO₂; wherein R^{c} is in each case independently selected from hydrogen, C₁₋₈ alkyl, C₃-C₈ cycloalkyl, C₂₋₈ heterocyclyl, C₆₋₁₂ aryl, C₁₋₁₂ heteroaryl, C₁₋₈ alkyl-C₃₋₈ cycloalkyl, C₁₋₈ alkyl-C₂₋₈ heterocyclyl, C₁₋₈ alkyl-C₆₋₁₂ aryl, and C₁₋₈ alkyl-C₃₋₁₂ heteroaryl;
wherein any two or more R₁ₐ, R^{1b}, R^{2a}, R^{2b}, R^{2c}, R^{2d}, R³ may together form a ring;
wherein any two or more R³, R^{4a} and R⁴, may together form a ring;
wherein any two or more R⁵, R^{4b} and R^{4d} may together form a ring;
wherein any two or more of R⁵ and R⁶ may together form a ring; and wherein any two or more of R⁶ and R^{7a}, R^{7b}, R^{7c}, R^{7d}, and R^{7c} may together form a ring.

In certain embodiments, the HDAC3 inhibitor comprises a compound of Formula (I) or a pharmaceutically acceptable salt, hydrate, solvate, or prodrug thereof, as described in WO2018119362A2 (incorporated herein by reference),

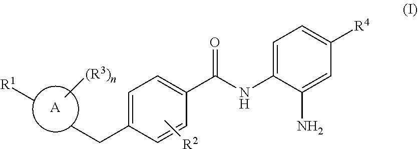

Wherein:
ring A is a 4-7 membered monocyclic heterocycloalkyi ring or a 7-12 membered spiro heterocycloalkyi ring, wherein ring A contains one nitrogen ring atom and optionally contains one additional ring atom independently selected from O, N, and S;
R¹ is H, C₁₋₆alkyl, C₂₋₆alkenyl, C₁₋₆hydroxyalkyl, C(O)C₁₋₆alkyl, C₀₋₃alkylene-C₃₋₁₀ cycloalkyl, or C₀₋₃alkylene-C₂₋₅heterocycloalkyl having 1 or 2 heteroatoms selected from O, S, N, and N(d₄alkyl);
R² is H, F, CI, or CH₃;
R³ is C₁₋₃alkyl;
R⁴ is H, F, or Cl; and
n is 0, 1, or 2,
optionally with the proviso that
   (a) ring A is not morpholino or thiomorpholino; and
   (b) when ring A is piperazinyl, R¹ is C₂₋₆alkenyl, C₁₋₆hydroxyalkyl, C(O)C₁₋₆alkyl, C₀₋₃alkylene-C₃₋₁₀cycloalkyl, or C₀₋₃alkylene-C₂₋₅cycloheteroalkyl having 1 or 2 heteroatoms selected from O, S, N, and N(C₁₋₄alkyl).

In certain embodiments, the HDAC3 inhibitor comprises a compound of Formula (I) or a pharmaceutically acceptable salt, hydrate, solvate, or prodrug thereof, as described in WO2014121062A1 (incorporated herein by reference),

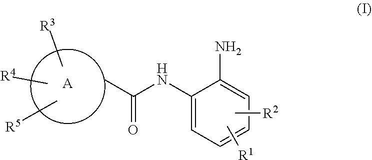

Wherein:
A is bicyclic heteroaryl or bicyclic heterocycloalkyl;
R¹ and R² are each independently selected from H or halo;
R is H, heterocycloalkyl, or wherein the heterocycloalkyl or C₁₋₆-alkyl-heterocycloalkyl groups are optionally substituted;
R⁴ and R⁵ are each independently selected from H, C₁₋₆-alkyl, C₂₋₆-alkenyl, C₂₋₆-alkynyl, C₃₋₆-cycloalkyl, C₁₋₆-alkyl-C₃₋₆-cycloalkyl, heterocycloalkyl, C₁₋₆-alkyl-heterocycloalkyl, NR⁶R⁷, O—C₁₋₆-alkyl-OR⁸, C₁₋₆-alkyl-OR⁸, aryl, C₁₋₆-alkyl-aryl, heteroaryl, C₁₋₆-alkyl-heteroaryl, C(O)N(R⁶)-heteroaryl, C(O)N(R⁶)-heterocycloalkyl, C(O)N(R⁶)-aryl, C(0)-NR⁶R⁷, C(0)-heteroaryl, C(O)-heterocycloalkyl, C(O)-aryl, C(O)—C₁₋₆-alkyl, CO₂—heteroaryl, CO₂-heterocycloalkyl, CO₂-aryl, CO₂-C₁₋₆-alkyl, or C(O)—C₁₋₆-alkyl-heterocycloalkyl, wherein the cycloalkyl, heterocycloalkyl, aryl, or heteroaryl groups are optionally substituted;
R⁶ and R⁷ are each independently selected from H, C₁₋₆-alkyl, C₁₋₆-alkyl-OR⁸, CO₂R₈, or C₁₋₆-alkyl-aryl; and
R⁸ is H or C₁₋₆-alkyl.

In certain embodiments, the HDAC3 inhibitor comprises a compound of Formula (I) or a pharmaceutically acceptable salt, hydrate, solvate, or prodrug thereof, as described in WO2014018979A1 (incorporated herein by reference),

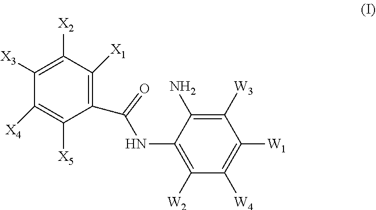

Wherein:
W₁, W₂, W₃, and W₄ are each independently selected from hydrogen, fluorine, chlorine, bromine, CF₃, CH₃, and deuterium, provided that at least one of W₁, W₂, W₃, or W₄ is not hydrogen;
X₁ and X₅ are each independently selected from hydrogen, halogen and C₁-C₃ alkyl;
X₂, X₃, and X₄ are each independently selected from hydrogen, halogen, OR⁵, C(O)R⁶, OS(O)ₚR⁷, NR³R₄, NR′C(O)R², NR′S(O)ₚR⁷, S(O)_{q}R¹⁰, C(O)ORⁿ, C(O)NR¹²R¹³, OC(O)OR¹⁴, OC(O)NR¹⁵R¹⁶, NR¹⁷C(O)OR¹⁸, NR¹⁹C(O)NR²⁰R²¹, C₁-C₈ alkyl, C₂-C₈ alkenyl, C₂-C₈ alkynyl, C₃-C₈ cycloalkyl, C₄-C₈ cycloaikenyl, aromatic ring, 3-8 membered heteroaromatic ring, and 3-8 membered saturated or partially saturated heterocyclic ring, wherein said alkyl, alkenyl, alkynyl, cycloalkyl, cycloaikenyl, aromatic ring, heteroaromatic ring, and heterocyclic ring is unsubstituted or substituted with one or more Rₐ and one or two of X₂, X₃, and X₄ is hydrogen;
Rₐ is selected from halogen, OR²⁵, C₁-C₈ alkyl, CF₃, CHF₂, CH₂F, NR²⁶C(O)R²⁷, and NR²⁸R²⁹; or
X₂ and X₃ or X₄ and X₃ taken together with the atoms to which they are attached form ring selected from a C₃-C₈ cycloalkyl, C₄-C₅ cycloaikenyl, aromatic ring, 3-8 membered heteroaromatic ring and 3-8 membered saturated or partially saturated heterocyclic ring, wherein said ring is unsubstituted or substituted with one or more Rv,
R^{v} is selected from halogen, OR²⁵, C₁-C₈ alkyl, CF₃, CHF₂, CH₂F, NR²⁶C(O)R²⁷, NR²⁸R²⁹, S(O)_{q}R⁷, S(O)_{q}R¹⁰, C(O)ORⁿ, C(O)NR¹²R¹³, OC(O)OR¹⁴, OC(O)NR¹⁵R¹⁶, NR¹⁷C(O)OR¹⁸, and NR¹⁹C(O)NR²⁰R²¹;
R¹ and R²⁶ are each independently selected from hydrogen and C₁-C₈ alkyl;
R² is selected from hydrogen, C₁-C₈ alkyl, C₂-C₈ alkenyl, C₃-C₈ alkynyl, C₃-C₈ cycloalkyl, C₄-C₅ cycloaikenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring, and heterocyclic ring are unsubstituted or substituted with one or more R^{b};
R²⁷ is selected from hydrogen, C₁-C₈ alkyl, C₃-C₈ alkynyl, C₃-C₈ cycloalkyl, C₄-C₅ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring, and heterocyclic ring are unsubstituted or substituted with one or more R^{b};
R^{b} is selected from halogen, C₁-C₈ alkyl, CF₃, CHF₂, CH₂F, OR²⁵, NH₂, NHCH₃, N(CH₃), C₃-C₈ cycloalkyl, C₄-C₅ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more R^{b1};
R^{b1} is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R³ and R⁴ are each independently selected from hydrogen, C₁-C₈ alkyl, C₂-C₈ alkenyl, C₃-C₈ alkynyl, C₃-C₈ cycloalkyl, C₄-C₅ cycloakenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubstituted or substituted with one or more R^{g};
R²⁸ and R²⁹ are each independently selected from hydrogen, C₁-C₈ alkyl, C₂-C₈ alkenyl, C₃-C₈ alkynyl, C₃-C₈ cycloalkyl, C₄-C₈ cycloakenyl, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubsituted or substituted with one or more R^{g};
R^{g} is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, and 3-8 membered saturated or partially saturated heterocyclic ring, wherein said cycloalkyl, cycloalkenyl, aromatic ring, heteroaromatic ring, and heterocyclic ring is unsubstituted or substituted with one or more R^{h};
R^{h} is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R⁵ and R²⁵ are each independently selected from hydrogen, C₁-C₈ alkyl, CF₃, CHF₂, CH₂F, C₂-C₈ alkenyl, C₃-C₈ alkynyl, C₃-C₈ cycloalkyl, C₄-C₈ cycloalkenyl, aromatic ring, 3-8 membered heteroaromatic ring, 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubsituted or substituted with one or more R^{c};
R^{c} is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more R^{d};
R^{d} is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R⁶ is selected from hydrogen, OR²⁵, C C₈ alkyl, CF₃, CHF₂, CH₂F, C₂-C₈ alkenyl, C₃-C₈ alkynyl, C₃-C₈ cycloalkyl, C₄-C₈ cycloalkenyl, aromatic ring, 3-8 membered heteroaromatic ring, and 3-8 membered saturated or partially saturated heterocyclic ring; wherein said alkyl, alkenyl, alkynyl, cycloalkyl, cycloalkenyl, aromatic ring, heteroaromatic ring and heterocyclic ring are unsubsituted or substituted with one or more R^{e};
R^{e} is selected from halogen, C,-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more R;
R^{f} is selected from halogen, C₁-C₃ alkyl, CF₃, CHF₂, CH₂F, OH, OCH₃, SOCH₃, S(O)₂CH₃, NH₂, NHCH₃, and N(CH₃)₂;
R⁷ is selected from C₁-C₈ alkyl, C₃-C₈ cycloalkyl, C₄-C₅ cycloalkenyl, aromatic ring, 3-8 membered heteroaromatic ring, and 3-8 membered saturated or partially saturated heterocyclic ring; wherein said alkyl, cycloalkyl, cycloalkenyl, aromatic ring, heteroaromatic ring, and heterocyclic ring are unsubstituted or substituted with one or more R¹;
R* is selected from halogen, C,-C₃ alkyl, CF₃, CHF₂, CH₂F, OH, OCH₃, NH₂, NHCH₃, and N(CH₃)₂; R are each independently selected from C₁-C₈ alkyl, C₃-C₈ cycloalkyl, C₄-C₅ cycloalkenyl, aromatic ring, 3-8 membered heteroaromatic ring, and 3-8 membered saturated or partially saturated heterocyclic ring; wherein said alkyl, cycloalkyl, cycloalkenyl, aromatic ring, heteroaromatic ring, and heterocyclic ring are unsubstituted or substituted with one or more R^{J};
R^{j} is selected from halogen, C,-C₃ alkyl, CF₃, CHF₂, CH₂F, OH, OCH₃, NH₂, NHCH₃, and N(CH₃)₂;
R¹¹ is selected from hydrogen, C₁-C₈ alkyl, C₂-C₈ alkenyl, C₂-C₈ alkynyl, C₃-C₈ cycloalkyl, C₄-C₅ cycloalkenyl, aromatic ring, 3-8 membered heteroaromatic ring, and 3-8 membered saturated or partially saturated heterocyclic ring; wherein said alkyl, alkenyl, alkynyl, cycloalkyl, cycloalkenyl, aromatic ring, heteroaromatic ring and heterocyclic ring are unsubstituted or substituted with one or more R^{k};
R^{k} is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₅ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more R^{k1};
R^{k1} is selected from halogen, C₁-C₃ alkyl, CF₃, CHF₂, CH₂F, OH, OCH₃, SO₂CH₃, NH₂, NHCH₃, and N(CH₃)₂;
R¹² and R¹³ are each independently selected from hydrogen, C₁-C₈ alkyl, C₂-C₈ alkenyl, C₃-C₈ alkynyl, C₃-C₈ cycloalkyl, C₄-C₅ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubsituted or substituted with one or more R¹;
R¹ is selected from halogen, C C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, and 3-8 membered saturated or partially saturated heterocyclic ring, wherein said cycloalkyl, cycloalkenyl, aromatic ring, heteroaromatic ring, and heterocyclic ring is unsubstituted or substituted with one or more R^{m};
R^{m} is selected from halogen, C C₃ alkyl, CF₃, CHF₂, CH₂F, OH, OCH₃, SOCH₃, S(O)₂CH₃, NH₂, NHCH₃, and N(CH₃)₂;
R¹⁴ is selected from C₁-C₈ alkyl, C₂-C₈ alkenyl, C₂-C₈ alkynyl, C₃-C₈ cycloalkyl, C₄-C₈cycloalkenyl, and aromatic ring; wherein said alkyl, alkenyl, alkynyl, cycloalkyl, cycloalkenyl, and aromatic ring are unsubstituted or substituted with one or more R″;
R″ is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more Rⁿ¹;
Rⁿ¹ is selected from halogen, C₁-C₃ alkyl, CF₃, CHF₂, CH₂F, OH, OCH₃, SO₂CH₃, NH₂, NHCH₃, and N(CH₃)₂;
R¹⁵ and R¹⁶ are each independently selected from hydrogen, C₁-C₈ alkyl, C₂-C₈ alkenyl, C₃-C₈ alkynyl, C₃-C₈ cycloalkyl, C₄-C₈ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubstituted or substituted with one or more R^{o};
R^{o} is selected from halogen, C₁-C₃ alkyl, CF₃, CHF₂, CH₂F, OH, OCH₃, NH₂, NHCH₃, N(CH₃)₂, C₃-C₈ cycloalkyl, C₄-Cg cycloalkenyl, aromatic ring, 3-8 membered heteroaromatic ring, and 3-8 membered saturated or partially saturated heterocyclic ring, wherein said cycloalkyl, cycloalkenyl, aromatic ring, heteroaromatic ring, and heterocyclic ring is unsubstituted or substituted with one or more R^{p};
R^{p} is selected from halogen, C₁-C₃ alkyl, CF₃, CHF₂, CH₂F, OH, OCH₃, SOCH₃, S(O)₂CH₃) NH₂, NHCH₃, and N(CH₃)₂;
R¹⁷ and R¹⁹ are each independently selected from hydrogen and C₁-C₈ alkyl;
R¹⁸ is selected from C₁-C₈ alkyl, C₂-C₈ alkenyl, C₂-C₈ alkynyl, C₃-C₈ cycloalkyl, C₄-C₈cycloalkenyl, aromatic ring, 3-8 membered heteroaromatic ring, and 3-8 membered saturated heterocyclic ring; wherein said alkyl, alkenyl, alkynyl, cycloalkyl, cycloalkenyl, heteroaromatic ring, heterocyclic ring, and aromatic ring are unsubstituted or substituted with one or more R^{q};
R^{q} is selected from halogen, C₁-C₃ alkyl, CF₃, CHF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, 3-8 membered saturated or partially saturated heterocyclic ring, wherein said cycloalkyl, cycloalkenyl, aromatic ring, heteroaromatic ring, and heterocyclic ring is unsubstituted or substituted with one or more R^{q1};
R^{q1} is selected from halogen, C₁-C₃ alkyl, CF₃, CHF₂, CH₂F, OH, OCH₃, SO₂CH₃, NH₂, NHCH₃, and N(CH₃)₂;
R²⁰ and R²¹ are each independently selected from selected from hydrogen, C₁-C₈alkyl, C₂-C₈ alkenyl, C₃-C₈ alkynyl, C₃-C₈ cycloalkyl, C₄-C₈ cycloalkenyl, aromatic ring, 3-8 membered heteroaromatic ring, and 3-8 membered saturated or partially saturated heterocyclic ring, wherein said alkyl, alkenyl, alkynyl, cycloalkyl, cycloalkenyl, aromatic ring, heteroaromatic ring and heterocyclic ring are unsubstituted or substituted with one or more Rᵣ;
Rᵣ is selected from halogen, C₁-C₃ alkyl, CF₃, CHCF₂, CH₂F, OH, OCH₃, NH₂, NHCH₃, N(CH₃)₂, C₃-C₈ cycloalkyl, C₄-C₈ cycloalkenyl, aromatic ring, 3-8 membered heteroaromatic ring, and 3-8 membered saturated or partially saturated heterocyclic ring, wherein said cycloalkyl, cycloalkenyl, aromatic ring, heteroaromatic ring, and heterocyclic ring is unsubstituted or substituted with one or more R^{s};
R^{s} is selected from halogen, C₁-C₃ alkyl, CF₃, CHF₂, CH₂F, OH, OCH₃, SOCH₃, SO₂CH₃, NH₂, NHCH₃, and N(CH₃)₂; and
p and q are each independently selected from 0, 1, and 2.

In certain embodiments, the HDAC3 inhibitor comprises a compound of Formula (I) or a pharmaceutically acceptable salt, hydrate, solvate, or prodrug thereof, as described in WO2010028193A1 (incorporated herein by reference),

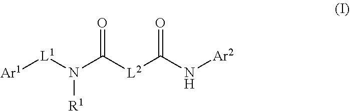

wherein:
R¹ is selected from H, C₁₋₄ alkyl, C₁₋₄ haloalkyl, C₁₋₄ alkoxycarbonyl, carbamyl, di-C₁₋₄-alkyl-carbamyl, and C₁₋₄ alkylcarbamyl;
Ar1 is selected from phenyl, 6-membered heteroaryl, and 5-membered heteroaryl, each of which is substituted by n independently selected Ry groups; wherein said phenyl, 6-membered heteroaryl, and 5-membered heteroaryl are each further optionally fused to a phenyl ring, which is optionally substituted by 1 or 2 groups independently selected from halogen, hydroxyl, cyano, nitro, C₁₋₄ alkyl, C₁₋₄ haloalkyl, C₁₋₄ alkoxy, C₁₋₄ haloalkoxy, amino, C₁₋₄ alkylamino, and di-C₁₋₄-alkylamino;
Ar2 is selected from phenyl and 6-membered heteroaryl; wherein said phenyl and 6-membered heteroaryl are each substituted at one ortho position by one J group and are each substituted by m independently selected Rz groups;
J is selected from hydroxyl and amino;
L1 is selected from a bond or C₁₋₄ alkylene, when Ar1 is an optionally substituted 6-membered heteroaryl or 5-membered heteroaryl; or L1 is a bond, when Ar1 is optionally substituted phenyl;
L2 is straight chain C₅-C₆ alkylene, wherein (i) the straight chain C₅-C₆ alkylene is optionally substituted by 1, 2, 3, or 4 independently selected Rx groups or (ii) one of the carbon atoms of the straight chain C₅-C₆ alkylene is replaced with —O—, provided that the carbon atom replaced with —O—is not the carbon atom that is directly attached to C(O)NHAr² or the carbon atom that is directly attached to C(O)NR¹-L¹-Ar¹;
or L2 is C₄-C₆ alkenylene, which is optionally substituted by 1, 2, 3, or 4 independently selected Rx groups;
each Rx is independently selected from halogen, hydroxyl, oxo, cyano, nitro, C₁₋₄ alkyl, C₁₋₄ alkoxy, C₁₋₄ haloalkyl, C₁₋₄ haloalkoxy, amino, C₁₋₄ alkylamino, and di-C₁₋₄-alkylamino;
each Ry is independently selected from halogen, cyano, nitro, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ alkoxycarbonyl, C₁₋₆ alkylcarbonyl, carbamyl, C₁₋₆ alkylcarbamyl, di-C₁₋₆ alkylcarbamyl, C₁₋₆ alkylcarbonylamino, C₁₋₆ alkylcarbonyl-(C₁₄-alkyl)amino, C₁₋₆ alkoxy carbonylamino, di-C₁₋₆ alkylamino, C₃₋₇cycloalkyl, C₂₋₆ heterocycloalkyl, phenyl, C₁₋₆ heteroaryl, C₃₋₇ cycloalkyl-C₁₋₄-alkyl, C₂₋₆ heterocycloalkyl-C₁₋₄-alkyl, phenyl-C₁₋₄-alkyl, and C₁₋₆ heteroaryl-C₁₋₄-alkyl; wherein said C₁₋₆ alkyl, C₂₋₆ alkenyl, C₂₋₆ alkynyl, C₁₋₆ haloalkyl, C₁₋₆ alkoxy, C₁₋₆ haloalkoxy, C₁_s alkoxycarbonyl, C₁₋₆alkylcarbonyl, carbamyl, C₁₋₆ alkylcarbamyl, di-C₁₋₆alkylcarbamyl, C₁₋₆ alkylcarbonylamino, C₁₋₆alkylcarbonyl-(C₁₋₄-alkyl)amino, C₁₋₆ alkoxy carbonylamino, and di-C₁₋₆ alkylamino are each optionally substituted by 1, 2, or 3 independently selected Ry groups; and wherein said C cycloalkyl, C₂₋₆ heterocycloalkyl, phenyl, C₁₋₆ heteroaryl, C₃₋₇ cycloalkyl-C₁₋₄-alkyl, C₂₋₆ heterocycloalkyl-C₁₋₄-alkyl, phenyl-C₁₋₄-alkyl, and C₁₋₆ heteroaryl-C₁₋₄-alkyl are each optionally substituted by 1, 2, or 3 independently selected Ry groups;
provided that only one of Ry is selected from optionally substituted C₃₋₇ cycloalkyl, C₂₋₆ heterocycloalkyl, phenyl, C₁₋₆ heteroaryl, C₃₋₇ cycloalkyl-C₁₋₄-alkyl, C₂₋₆ heterocycloalkyl-C₁-4-alkyl, phenyl-C₁-4-alkyl, and C₁₋₆ heteroaryl-C₁-4-alkyl;
each Rz is independently selected from halogen, cyano, nitro, hydroxyl, C₁₋₆ alkyl, C₂₋₆alkenyl, C₂₋₆ alkynyl, C₁₋₆ haloalkyl, C₁₋₆ alkoxy, C₁₋₆ haloalkoxy, C₁₋₆ alkoxycarbonyl, C₁_s alkylcarbonyl, carbamyl, C₁₋₆ alkylcarbamyl, di-C₁₋₆ alkylcarbamyl, C₁₋₆ alkylcarbonylamino, C₁₋₆ alkylcarbonyl-(C₁₄-alkyl)amino, C₁₋₆ alkoxy carbonylamino, amino, C₁₋₆ alkylamino, di-C₁₋₆ alkylamino, C₃₋₇ cycloalkyl, C₂₋₆ heterocycloalkyl, phenyl, and C₁₋₆ heteroaryl;
wherein said C₁₋₆ alkyl, C₂₋₆ alkenyl, C₂₋₆ alkynyl, C₁₋₆ haloalkyl, C₁₋₆ alkoxy, C₁_s haloalkoxy, C₁₋₆ alkoxycarbonyl, C₁₋₆ alkylcarbonyl, carbamyl, C₁₋₆ alkylcarbamyl, di-C₁₋₆ alkylcarbamyl, C₁₋₆ alkylcarbonylamino, C₁₋₆ alkylcarbonyl-(C₁-4-alkyl)amino, C₁₋₆ alkoxy carbonylamino, C₁₋₆ alkylamino, and di-C₁₋₆ alkylamino are each optionally substituted by 1, 2, or 3 independently selected Rz groups; and
wherein said C₃₋₇ cycloalkyl, C₂₋₆ heterocycloalkyl, phenyl, and C₁₋₆ heteroaryl are each optionally substituted by 1, 2, or 3 independently selected Rz groups;
provided that only one of Rz is selected from optionally substituted C₃₋₇ cycloalkyl, optionally substituted C₂₋₆ heterocycloalkyl, optionally substituted phenyl, and optionally substituted C₁₋₆ heteroaryl;
each Ry and Rz is independently selected from hydroxyl, cyano, nitro, C₁₋₄ alkoxy, C₁₋₄ haloalkoxy, amino, C₁₋₄ alkylamino, and di-C₁₋₄-alkylamino;
each Ry and Rz is independently selected from halogen, hydroxyl, cyano, nitro, C₁_s alkyl, C₁₋₄ haloalkyl, C₁₋₄ alkoxy, C₁₋₄ haloalkoxy, amino, C₁₋₄ alkylamino, and di-C₁₄-alkylamino;
n is an integer selected from 0, 1, 2, 3, and 4; and
m is an integer selected from 0, 1, 2, and 3;
provided that when L2 is straight chain C alkylene, then m 1, 2, and 3, and one occurrence of Rz is optionally substituted phenyl or optionally substituted C₁₋₆ heteroaryl.

Additional suitable HDAC3 inhibitors include those in WO2016018795A1, WO2015200699A2, US20150359794A1, WO2015069810A1, WO2012118782A1, WO2014143666A1, WO2013005049A1, WO2009045440A1, WO2007022041A2, all incorporated herein by reference.

In certain embodiments, the HDAC3 inhibitor is specific or selective for HDAC3 (e.g., having IC50 of at least about 2-fold, 5-fold, 10-fold, 20-fold, 50-fold, 100-fold, 200-fold, 500-fold, 1000-fold or more compared to IC50 for a non-HDAC3, such as a Class 2 or Class 3 HDAC, or HDAC1, HDAC2, or HDAC8).

In certain embodiments, the HDAC3 inhibitor comprises an antisense oligonucleotide capable of hybridizing with a nucleic acid molecule encoding HDAC3 protein, wherein the oligonucleotide inhibits the expression of HDAC3 protein.

In certain embodiments, the HDAC3 inhibitor comprises: (a) a Cas effector enzyme such as Cas9 protein, or a polynucleotide encoding thereof; and (b) a CRISPR-Cas system guide RNA polynucleotide specific for HDAC3.

In certain embodiments, the HDAC3 inhibitor comprises a proteolysis-targeting chimera (PROTAC) that targets HDAC3 for proteasome-mediated degradation.

In certain embodiments, the HDAC3 inhibitor comprises an siRNA, shRNA, or RNAi reagent specific for HDAC3.

### EXAMPLES

### Example 1 Identification of HDAC Inhibitors as Inhibitors to Suppress Formation of Damaging Reactive Astrocytes

Damaging reactive astrocytes (DRA) are present in many neurological diseases. Applicant has developed a proprietary in vitro culture system to culture astrocytes in their resting state, and has successfully induced the formation of damaging reactive astrocytes from the resting astrocytes in vitro, to mimic this pro-inflammatory toxic state in vivo, through the exogenous delivery of proinflammatory cytokines such as Tumor Necrosis Factor-alpha (TNFα), interleukin 1-alpha (IL-1α), and complement component 1q (C1q). Exposure of the cultured resting astrocytes to these factors resulted in the formation of damaging reactive astrocytes that exhibited a cell state-specific gene expression signature.

This example demonstrates that such an in vitro culture system can be used to identify chemical compounds capable of blocking the induction/formation of DRA from resting astrocytes, and use the identified inhibitors to treat diseases associated with the DRA.

Specifically, resting astrocytes were first thawed and plated onto 384-well plates at a density of about 500 cells/mm². A Perkin Elmer Janus G3 Varispan Automated Workstation was then used to treat cells with candidate small-molecules from a library of 3115 bioactive small-molecules, with one type of small molecule per well, at a concentration of about 2 μM in 384-well plates, followed one hour later by exposing the pre-treated astrocytes with such reactive astrocyte driving factors as IL-1α (3 ng/mL, Sigma #I3901), C1q (400 ng/mL, MyBioSource #MBS143105), and TNFα (30 ng/mL, R&D Systems #210-TA-020).

After incubation for about 24 hours, the cells were fixed using 4% paraformaldehyde and stained for GBP2 (Proteintech #11854-1-AP) using the procedure detailed in the immuno-cytochemistry section below. Here, GBP2 is an interferon-inducible GTPase that is up-regulated in both mouse and human reactive astrocytes, and has been associated with a damaging reactive astrocyte state. In addition, GBP2 protein levels displayed consistently high signal to background ratio in resting vs reactive astrocytes across all screening plates, making it a suitable phenotypic readout for a high-throughput screen.

The cells were then imaged using the PerkinElmer Operetta CLS High-Content Analysis System. Images were analyzed using automated PerkinElmer Columbus Image Analysis Software.

For analysis, toxic chemicals were first removed. A chemical was considered toxic if it decreased the counted number of live cells in the well by greater than 30% compared to reactive astrocyte plus DMSO vehicle control wells.

Hits were then determined as those compounds that decreased the number of GBP2⁺ reactive astrocytes by greater than 80% compared to reactive astrocytes plus DMSO vehicle control wells.

This primary screen had high reproducibility across independent compound plates and a z-prime score of 0.734, which is excellent for a cell-based screen. This robust primary screen resulted in the discovery of 93 potential inhibitors of damaging reactive astrocyte polarization. These 93 primary hits were filtered using a secondary 8 point dose curve screen with validate compounds successfully decreasing GBP2⁺ reactive astrocytes over at least 3 different concentrations. The result of this dose curve screen is the validation of 33 compounds across 14 compound target classes that all inhibit the formation of damaging reactive astrocytes polarized by TNFα, IL1α, and C1q. None of these compounds have been previously described for this function.

Notably, the validated small-molecule inhibitors of reactive astrocyte formation were enriched for histone deacetylase (HDAC) inhibitors, and inhibitors of NFκB signaling (data not shown). Representative HDAC inhibitors identified in the phenotypic screening were listed in Table 1 below. Also see FIG. 1.

TABLE 1
Representative HDAC inhibitors Identified by
our Primary High-Throughput Screen to Inhibit
the Formation of Damaging Reactive Astrocytes

| | |
|---|---|
| Name of | |
| Hits | Description |

Pan-HDAC Inhibitors (inhibit members of more than one HDAC classes)

| | |
|---|---|
| AR-42 | AR-42 (HDAC-42) is a pan-HDAC inhibitor with IC50 |
| | of 30 nM. |
| Belinostat | Belinostat (PXD101, NSC726630, PX-105684) is a pan- |
| | HDAC inhibitor with IC50 of 27 nM in a cell-free |
| | assay, with activity demonstrated in cisplatin- |
| | resistant tumors. Belinostat induces autophagy. |
| Givinostat | Givinostat (ITF2357) is an HDAC2 inhibitor. It is |
| | a potent HDAC inhibitor for maize HD2, HD1B and |
| | HD1A with IC50 of 10 nM, 7.5 nM and 16 nM in |
| | cell-free assays. |
| Dacinostat | Dacinostat (LAQ824, NVP-LAQ824) is a pan-HDAC |
| | inhibitor with IC50 of 32 nM, and is known to |
| | activate the p21 promoter. |
| M344 | M344 is a potent pan-HDAC inhibitor with IC50 of |
| | 100 nM, and is able to induce cell differentiation. |
| Panobinostat | Panobinostat (LBH589, NVP-LBH589) is a broad- |
| | spectrum HDAC inhibitor with IC50 of 5 nM in a |
| | cell-free assay. Panobinostat (LBH589) induces |
| | autophagy and apoptosis. Panobinostat effectively |
| | disrupts HIV latency in vivo. |
| Abexinostat | Abexinostat (PCI-24781, CRA-024781) is a pan-HDAC |
| | inhibitor mostly targeting HDAC1 with Ki of 7 nM, |
| | modest potent to HDACs 2, 3, 6, and 10 and greater |
| | than 40-fold selectivity against HDAC8. |
| Pracinostat | Pracinostat (SB939) is a pan-HDAC inhibitor with |
| | IC50 of 40-140 nM with exception for HDAC6. It has no |
| | activity against the class III isoenzyme SIRT I. |
| | Pracinostat induces apoptosis in tumor cells. |
| Quisinostat | Quisinostat (JNJ-26481585) is a pan-HDAC inhibitor. |
| | Quisinostat•2HCl is a second-generation HDAC |
| | inhibitor with highest potency for HDAC1 with IC50 |
| | of 0.11 nM in a cell-free assay, modest potent to |
| | HDACs 2, 4, 10, and 11; greater than 30-fold |
| | selectivity against HDACs 3, 5, 8, and 9 and lowest |
| | potency to HDACs 6 and 7. |
| Rocilinostat | Rocilinostat (ACY-1215) is a selective HDAC6 inhibitor |
| | with IC50 of 5 nM in a cell-free assay. It is >10- |
| | fold more selective for HDAC6 than HDAC1/2/3 (class I |
| | HDACs) with slight activity against HDAC8, minimal |
| | activity against HDAC4/5/7/9/11, Sirtuin1, and |
| | Sirtuin2. Ricolinostat suppresses cell proliferation |
| | and promotes apoptosis. |
| Scriptaid | Scriptaid is a pan-HDAC inhibitor. It shows a |
| | greater effect on acetylated H4 than H3. |
| Trichostatin A | Trichostatin A (TSA) is a pan-HDAC inhibitor with |
| | IC50 of ~1.8 nM in cell-free assays, with the |
| | exception of HDAC8. |
| Vorinostat | Vorinostat (suberoylanilide hydroxamic acid, SAHA, |
| | MK0683) is a pan-HDAC inhibitor with IC50 of ~10 |
| | nM in a cell-free assay. Vorinostat abrogates |
| | productive HPV-18 DNA amplification. |

Class I (HDAC1/2/3/8) HDAC Inhibitors

| | |
|---|---|
| BG45 | BG45 is a class I HDAC inhibitor with IC50 of 289 |
| | nM, 2.0 μM, 2.2 μM and >20 μM for HDAC3, HDAC1, |
| | HDAC2, and HDAC6 in cell-free assays, respectively. |

HDAC3-Specific Inhibitors

| | |
|---|---|
| RGFP966 | RGFP966 is an HDAC3 specific inhibitor, with IC50 |
| | of 0.08 μM in cell-free assay, exhibits >200-fold |
| | selectivity over other HDAC. |
| BRD3308 | BRD3308 is a potent and highly selective inhibitor |
| | of HDAC3, with IC50 of 54 nM, 1.26 μM and 1.34 μM |
| | for HDAC3, HDAC1 and HDAC2, respectively. |
| | BRD3308 activates HIV-1 transcription. BRD3308 |
| | suppresses pancreatic β-cell apoptosis induced |
| | by inflammatory cytokines (glucolipotoxic stress) |
| | and increases functional insulin release. |
| HDAC3-IN- | HDAC3-IN-T247 is a potent and selective HDAC3 |
| T247 (T247) | inhibitor with a cell free IC50 of 55 nM. It acts |
| | by increasing NF-κB acetylation, inducing growth |
| | inhibition of cancer cells, and activating HIV gene |
| | expression in latent HIV-infected cells. (Suzuki |
| | et al. PloS One 2013) |

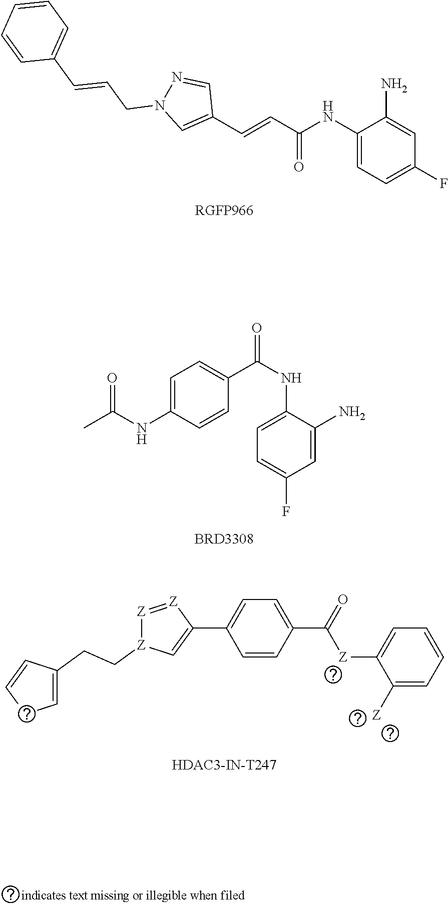

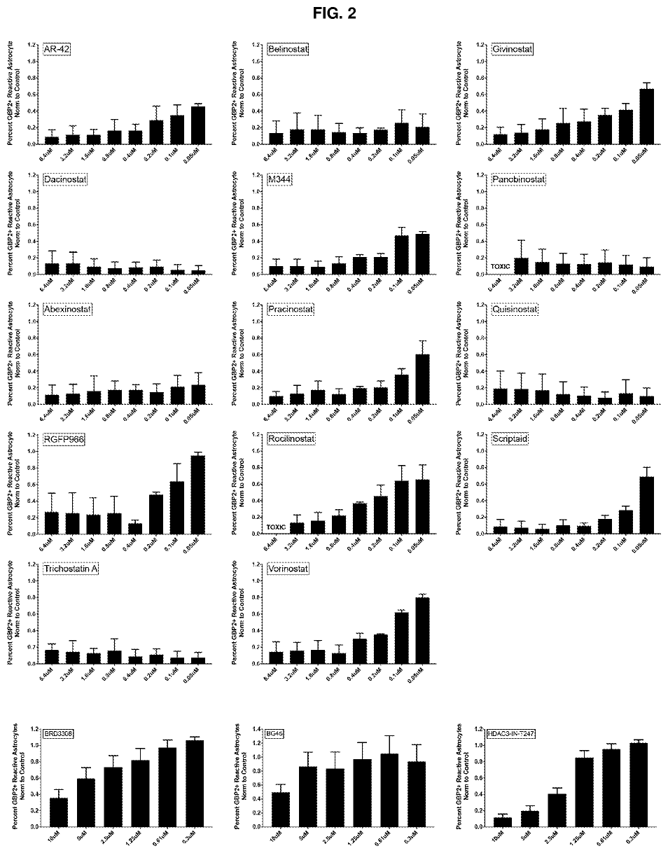

Like other primary hits, HDAC inhibitors that were discovered to be effective in the primary screen were tested at multiple doses. Dose curve analysis was run using the same liquid handling machinery and steps as for the primary screen. Eight total doses were tested, starting at 6.4 μM as the highest concentration, and decreasing by half-steps down to 0.05 μM. All HDAC inhibitors were effective at blocking the formation of GBP2⁺ damaging reactive astrocytes at multiple doses (FIG. 2).

HDAC inhibitors were then tested for their ability to block the acquisition of reactive astrocyte functional characteristics.

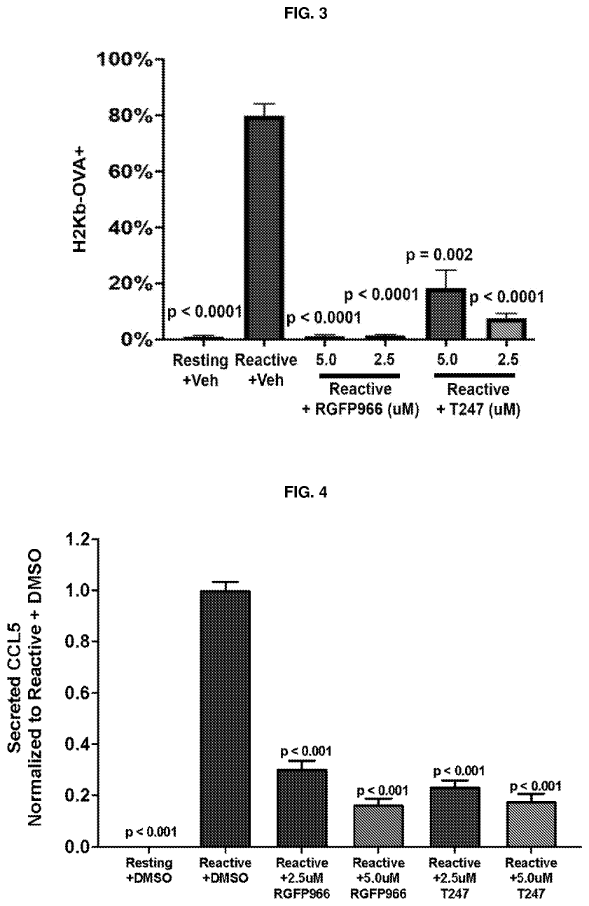

To test whether HDAC inhibitors block the ability of reactive astrocytes to cross present exogenous antigen, cells were plated down in 96-well plates at a density of about 500 cells/mm². Cells were then treated with HDAC inhibitors for 1 hr prior to the addition of reactive astrocyte driving factors as the concentrations stated above. Cells were incubated for 24 hrs, after which, media was washed out and replaced with media containing the peptide fragment ovalbumin 257-264 (OVA257-264). Cells were then incubated overnight, and then stained for 2 hrs at 37° C. with APC-conjugated antibody against MHC Class I (H-2Kb) bound to OVA257-264, then visualized. The percent of H-2Kb positive cells as a percent of total live cells was determined (FIG. 3).

It is apparent that more than about 80% of the reactive astrocytes were H-2Kb-OVA positive, while virtually none of the resting astrocytes were (p<0.0001). Stunningly, at either 2.5 or 5.0 μM, the HDAC3-specific inhibitor RGFP966 completely inhibited the acquisition of the damaging reactive astrocytes (DRAs) (p<0.0001) associated function of antigen cross-presentation by MHC Class I. A second HDAC3-specific inhibitor T247 had similar effects though to a slightly lesser degree (i.e., about 20% of treated cells were H-2 kB-OVA positive DRAs at about 5.0 μM, p<0.002, and about 8% of treated cells were H-2 kB-OVA positive DRAs at about 2.5 μM, p<0.0001).

HDAC inhibitors were additionally tested for their ability to block the secretion of CCL5 by reactive astrocytes.

Astrocytes were plated and treated exactly as described for the antigen cross presentation assay. However, after 24 hrs of incubation with either reactive astrocyte driving factors alone or with HDAC inhibitor treatment, conditioned media from the cells was collected and assayed for CCL5 using a commercially available colorimetric ELISA kit (RnD Cat #: DY478-05). Data was then fit to a standard four parameter logistic (4PL) curve and presented as pg/mL/cell, normalized to reactive astrocytes plus DMSO vehicle (FIG. 4).

Again, 2.5 μM of the HDAC3-specific inhibitor RGFP966 statistically significantly (p<0.001) reduced CCL5 secretion by about 70% compared to vehicle control, while 5.0 μM of RGFP966 reduced CCL5 secretion by more than 80% (p<0.001) (FIG. 4). Similar results were also observed for the HDAC3-specific inhibitor T247 at both concentrations (p<0.001 in both cases) (FIG. 4).

The data demonstrated that the phenotypic drug screening used here is a powerful tool to identify small-molecules and their target pathways that modulate cell state, and that one class of such identified small molecule inhibitors—the HDAC3-specific inhibitors—inhibit the formation of DRAs from resting astrocytes upon stimulation by DRA driving cytokines.

### Example 2 HDAC Inhibitors Block the Formation of Reactive Astrocytes In Vivo

This example demonstrates that HDAC inhibition blocks the formation of reactive astrocytes in vivo in a systemic lipopolysaccharide (LPS) injection mouse model to drive neuroinflammation and the formation of reactive astrocytes in the brain.

Mice at 7 weeks of age were injected i.p. with either RGFP966 vehicle (30% hydroxypropyl-β-cyclodextrin, 0.1 M sodium acetate, and 10% DMSO) or 10 mg/kg RGFP966 daily for 11 days. After which, mice were injected i.p. daily for two days with either LPS vehicle (saline) plus RGFP966 vehicle, 5 mg/kg LPS plus RGFP966 vehicle, or 5 mg/kg LPS plus 10 mg/kg RGFP966. After which, brain tissue was collected from the mice and processed for in situ hybridization with probes targeting GFAP as a general astrocyte marker, and the reactive astrocyte-specific marker GBP2.

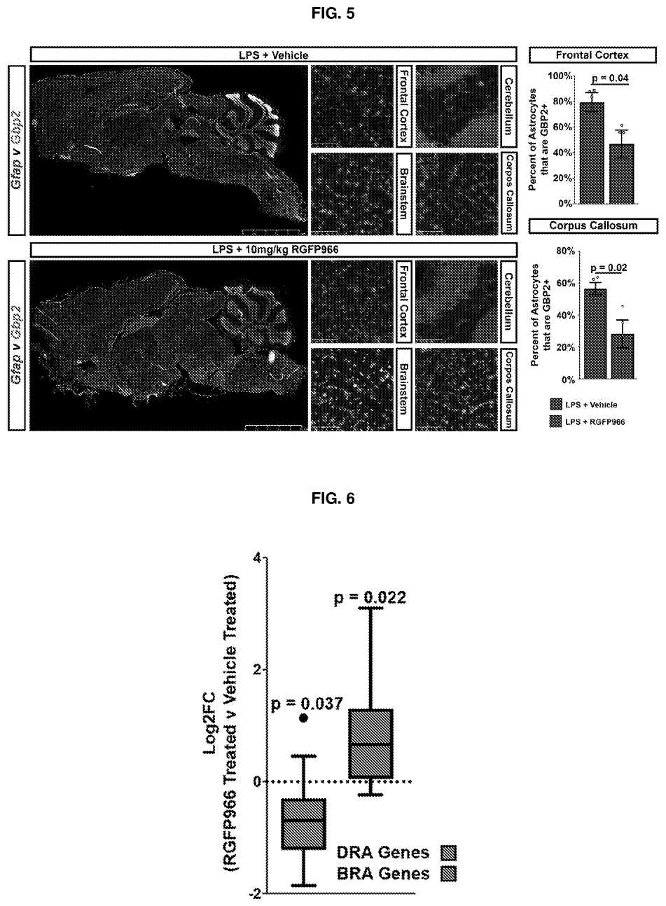

Mice treated with the HDAC inhibitor RGFP966 and LPS had statistically significantly (p<0.05) lower percentage (about half) of GFAP⁺ astrocytes expressing GBP2 in the frontal cortex (p=0.04) and corpus callosum (p=0.02), compared to vehicle plus LPS treated mice (FIG. 5). The data demonstrated that the HDAC3-specific inhibitor RGFP966 is not only able to inhibit the formation of DRAs in vitro, but also in vivo.

### Example 3 Epigenetic Regulation of Reactive Astrocyte Cell State Change

This example provides mechanistic understanding of how HDAC inhibitors, such as HDAC3 inhibitors, may inhibit the induction/formation of damaging reactive astrocytes. While not wishing to be bound by any particular theory, the data presented herein is consistent with the theory that astrocytes undergo epigenetic changes due to chromosomal remodeling when transitioning to DRAs, and blocking the required epigenetic changes via HDAC inhibitors may inhibit DRA formation.

In contrast to temporary changes in gene expression, cell state changes are driven by chromatin landscape remodeling that involves opening of previously closed genomic regions, resulting in sustained changes to gene expression and function. These chromatin changes include the loss and gain of super enhancers, regions of densely packed active enhancers identified by large deposits of the histone mark H3K27ac, a histone mark of active chromatin. This phenomenon has been extensively characterized during cell state changes in cancer, but has not been fully utilized to understand reactive astrocyte cell state changes.

To determine whether changes in the chromatin landscape occur during the transition to a reactive astrocyte state, chromatin immunoprecipitation sequencing (ChIPseq) was performed for H3K27ac in reactive and resting astrocytes.

Super enhancer analysis showed that, compared to resting astrocytes, reactive astrocytes gain 347 specific super enhancers and lose 324 super enhancers, while 484 super enhancer sites remain unchanged as astrocytes become reactive (data not shown). Pairing this H3K27ac data with an assay for transposase-accessible chromatin using sequencing (ATACseq), enriched transcription factor motifs were identified as reactive astrocyte gained super enhancers.

It was found that binding motifs for the p65/RelA subunit of NFκB, a known driver of neuroinflammation, were enriched in reactive astrocytes gained super enhancers, and absent from those that have lost super enhancers (data not shown). In agreement with the role of super enhancers as drivers of gene expression, the genes associated with gained super enhancers were significantly up-regulated in reactive astrocytes compared to resting astrocytes, whereas genes associated with lost super enhancers were down-regulated (data not shown).

These findings show that reactive astrocyte cell state change is driven in part by reorganization of the chromatin landscape and the gain of super enhancers. The data is consistent with the theory that blocking this chromatin reorganization may inhibit reactive astrocyte formation.

### Example 4 HDAC3-Regulated NF-κB Signaling is Required for Reactive Astrocyte Formation

In the phenotypic screen described in Example 1, it was found that as a compound class, HDAC inhibitors were significantly enriched, in that HDAC inhibitors represented 42.42% (14/33) of the validated hits, while all HDAC inhibitors in the library of 3100⁺compounds screened only accounted for about 0.95% (30/3139) of the total small molecules in the library.

While most of the HDAC inhibitors were non-specific, one validated HDAC inhibitor, RGFP966, is HDAC3-specific, with a cell-free IC50 of 0.08 PM, and no inhibition of other HDAC isozymes at up to 15 μM.

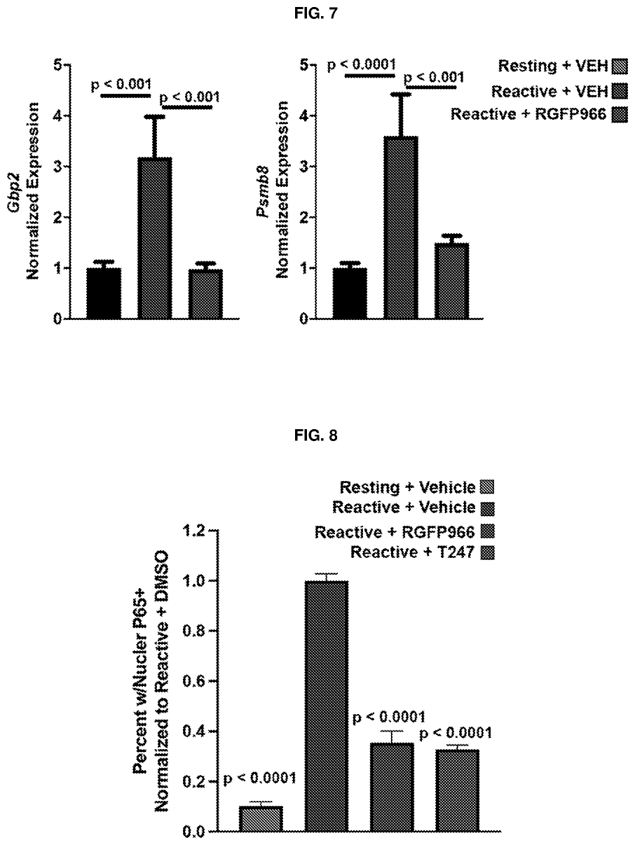

This data promoted further exploration into the role of HDAC3 in reactive astrocyte formation. Interestingly, bulk RNAseq analysis showed that HDAC3 inhibition by RGFP966 treatment decreased expression of genes associate with damaging reactive astrocytes (DRA), while increasing expression of genes associated with beneficial reactive astrocytes (BRA) (FIG. 6). Treatment with the HDAC3 specific inhibitor RGFP966 also decreased expression of genes associated with DRAs in cultured human astrocytes (FIG. 7). This data suggested that HDAC3 plays a role as a molecular switch between distinct reactive astrocyte subtypes.

Further, HDAC3 inhibition by RGFP966 decreases RelA/P65 driven transcription. This effect was driven, at least in part, by decreased nuclear translocation of RelA/P65 in reactive astrocytes treated with RGFP966. This was confirmed with a second HDAC3-specific inhibitor T24710 that also decreased nuclear RelA/P65 (FIG. 8). Decreased nuclear RelA/P65 led to a decrease in H3K27ac at RelA/P65 target genes, and specifically decreased expression of RelA/P65 target genes up-regulated in reactive astrocytes.

Further experiments showed that HDAC3 inhibition (e.g., by RGFP966 or T247) inhibited the acquisition of reactive astrocyte functions. Both HDAC3 inhibitors were able to block the ability of reactive astrocytes to process and present exogenous OVA₂₅₇₋₂₆₄ (FIG. 3).

In summary, the phenotypic screen (Example 1) successfully identified a role for an HDAC3-RelA/P65 signaling nexus in the formation and function of reactive astrocytes, the inhibition of which can be used to treat diseases (such as NDs) associated with reactive astrocyte formation.

### Example 5 RelA/P65 is a Key Molecular Regulator of Reactive Astrocyte State and Function

Both our super enhancer analysis (Example 3) and phenotypic screen (Example 1) revealed a role for RelA/P65 in reactive astrocyte state change. This example demonstrated that the validated small molecule inhibitors of reactive astrocyte state change inhibit RelA/P65 driven transcription, and supported the theory that inhibition of RelA/P65 is a unifying mechanism for our small-molecule inhibitors of reactive astrocyte formation.

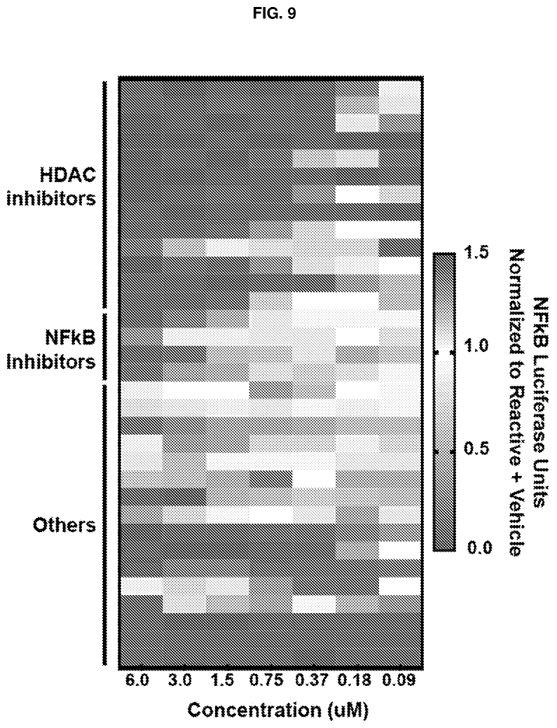

The data presented above showed that the majority of the validated small molecule inhibitors of reactive astrocyte formation (i.e., 27 out of 33) blocked RelA/P65 transcriptional activity (FIG. 9). Given the importance of NFκB as a regulator of the cellular response to inflammatory cytokines, the first p65/RelA ChIPseq was performed in reactive and resting astrocytes. It was found that RelA/P65 DNA binding was almost completely absent in resting astrocytes, but significantly increased as astrocytes transition to a reactive state (data not shown).

In agreement with its role as a transcriptional activator, the majority (142/229) of the RelA/P65 target genes in reactive astrocytes were upregulated, while fewer (12/229) genes were down-regulated or unchanged (75/229) (data not shown). Up-regulated RelA/P65 target genes included genes associated with damaging reactive astrocytes like C₃ and H2-D1, and were enriched for genes involved in immune and inflammation processes.

These results show that the NF-κB subunit RelA/P65 is a key regulator of reactive astrocyte state change and function, and a critical molecular target for developing reactive astrocyte focused therapeutics.

### Example 6 Pharmacological Inhibition of HDAC3 Promotes Tissue Repair in vivo

This example demonstrates that inhibition of HDAC3 promotes tissue repair by blocking the formation of reactive astrocytes in vivo.

First, it was confirmed that the HDAC3-specific inhibitor RGFP966 is brain permeant, and that with chronic treatment, a concentration in brain tissue equal to the IC50 of RGFP966 was reached (data not shown).

Systemic injections of LPS leads to robust neuroinflammation where astrocytes transition to a damaging, pro-inflammatory, reactive astrocyte state. The data presented herein demonstrates that HDAC3 inhibition by RGFP966 could block the formation of reactive astrocytes in mice challenged with systemic lipopolysaccharide (LPS) injections.

Specifically, young adult mice underwent chronic daily treatment of 10 mg/kg RGFP966, followed by two days of 5 mg/kg LPS injections. It was first confirmed that inhibition of HDAC3 with RGFP966 increased the levels of acetylated histone 4 (AcH4), providing evidence of target engagement in vivo (data not shown).

Next, RNAscope in situ hybridization was used to show that RGFP966 treatment blocked the formation of reactive astrocytes in LPS challenged mice. Mice that underwent chronic RGFP966 treatment prior to LPS challenge had decreased levels of GBP2⁺ reactive astrocytes in the frontal cortex, cerebellum, brainstem, and corpus callosum (FIG. 5). This result proved that HDAC3 inhibition by RGFP966 blocks reactive astrocyte formation in vivo.

Further data showed that blocking reactive astrocyte formation, by pharmacologically inhibiting HDAC3, could promote tissue repair. This was based on a toxin-based model of CNS tissue damage, where lysolecithin (lysophosphatidylcholine (LPC)) is injected into the dorsal column of the spinal cord leading to cell death, demyelination, and axonal damage at 4 days post lesion (dpl). This model undergoes spontaneous repair that is not widespread until 14-21 dpl, providing an opportunity to test whether small-molecules promote accelerated tissue repair.

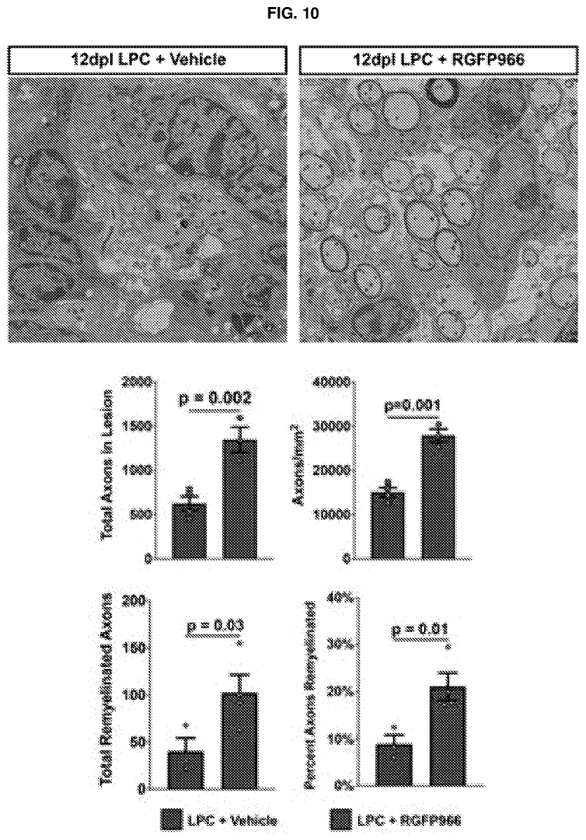

Compared to vehicle-treated LPC mice, animals that were injected daily with 10 mg/kg RGFP966 beginning at −1 dpl showed accelerated tissue repair at 12 dpl (FIG. 10). This was evident in the increase of total remyelinated and percentage of axons remyelinated in the lesion of LPC injected mice (FIG. 10).

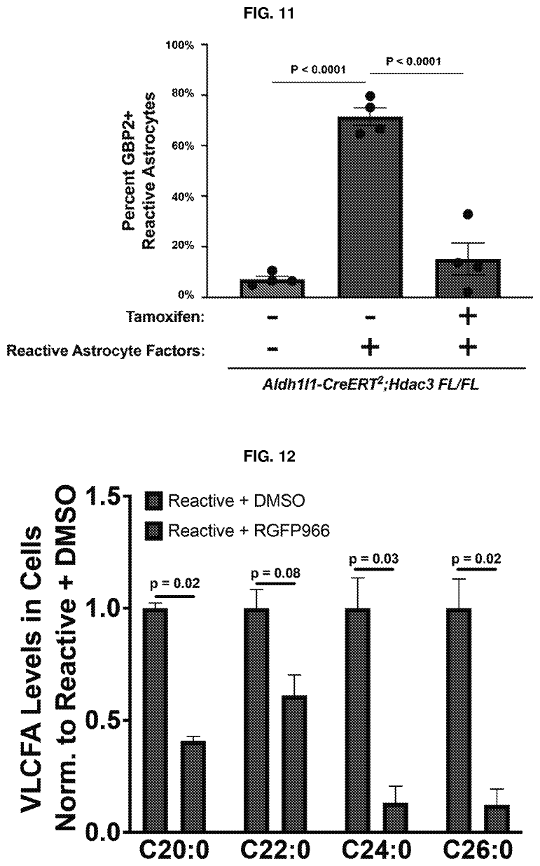

Primary astrocytes generated from tamoxifen inducible Hdac3 knockout mice were generated. Conditional Hdac3 knockout cells that were not treated with tamoxifen or reactive astrocyte factors (TNFα, IL1α, and C1q) did not become GBP2⁺ reactive astrocytes (FIG. 11). Conditional Hdac3 knockout cells that were not treated with tamoxifen, meaning Hdac3 remained expressed and functional, that were then treated with reactive astrocyte factors became GBP2⁺ reactive astrocytes. In contrast, conditional Hdac3 knockout astrocytes that were treated with tamoxifen, which activates the genetic excision of Hdac3, followed by reactive astrocyte factors did not become GBP2⁺ reactive astrocytes (FIG. 11). This genetically confirms that HDAC3 is required for GBP2⁺ reactive astrocyte formation.

Accumulation of VLCFA is known to be toxic to cells in the brain, and reactive astrocytes can be a source of VLCFA. Cells treated with HDAC inhibitor RGFP966 showed decreased levels of toxic very long chain fatty acids (VLCFA) when compared to cells treated with DMSO as a control (FIG. 12).

This data showed that inhibiting HDAC3 in astrocytes favors the formation of a reparative cellular environment following in vivo tissue damage, leading to accelerated tissue repair.

Reactive astrocytes play diverse roles in CNS injury and disease. Subtypes of reactive astrocytes can contribute to disease progression by exacerbating tissue damage. The data presented herein demonstrated that inhibiting the formation of these reactive astrocyte subtypes is a promising therapeutic approach.

Specifically, an HDAC3-NFκB signaling axis was identified as being required for the formation of reactive astrocytes in response to cytokines shown to drive the formation of a damaging reactive astrocyte state. Perturbing that signaling axis through pharmacological HDAC3 inhibition blocks the formation of reactive astrocytes in vitro, and is capable of promoting tissue repair following focal toxin induced injury in vivo.

These findings support the use of HDAC3 inhibitors as novel astrocyte-targeted therapies for treating neurodegenerative diseases.

Certain materials and methods used in one or more of the examples herein are provided below for illustrative purpose only, and is in no way limiting.

### Isolation and Generation of Resting Astrocytes

Brains from mice of the C57BL/6 strain were extracted at postnatal day 2 (P2), meninges were removed and cortices isolated. These cortices were dissociated following the protocols found in the Miltenyi Tumor Dissociation Kit (130-095-929, Miltenyi). After dissociation, cells were plated in flat-bottomed plastic flasks coated with a substrate of poly-L-ornithine (Sigma, P3655) and laminin (Sigma, L2020). Cells were cultured for 24 hours in media consisting of Dulbecco's Modified Eagle Medium/Nutrient Mixture F-12 (DMEM/F-12; 11320033, ThermoFisher Scientific), N-2 Supplement (17502048, ThermoFisher Scientific), B-27 Supplement (17504044, ThermoFisher Scientific), GlutaMAX Supplement (35050079, ThermoFisher Scientific), Penicillin-Streptomycin (15070063, ThermoFisher Scientific), and FGF-2. After 24 hours, the cells were switched to astrocyte enrichment media comprised of DMEM (11960044, ThermoFisher Scientific), Neurobasal Medium (21103049, ThermoFisher Scientific), GlutaMAX Supplement, Sodium Pyruvate (11360070, ThermoFisher Scientific), N-2max Supplement (R&D #AR009), N-acetyl cysteine (Sigma #A8199), Penicillin-Streptomycin (ThermoFisher Scientific #15070-063), 5 ng/mL HB-EGF (R&D Systems #259-HE-050), 10 ng/mL CNTF (R&D Systems #557-NT-010), 10 ng/mL BMP4 (R&D Systems #314-BP-050), and 20 ng/mL FGF2 (R&D Systems #233-FB-01M) to proliferate. Media was changed every 48 hours. Once confluent astrocytes were either cryopreserved or passaged, grown to confluency and then cryopreserved. To conduct a terminal experiment, cryopreserved astrocytes were removed from liquid nitrogen storage and thawed in astrocyte maturation media (DMEM, Neurobasal Medium, GlutaMAX Supplement, Sodium Pyruvate, N-2 Supplement, N-acetyl cysteine) supplemented with 5 ng/mL HB-EGF, 10 ng/mL CNTF, 50 ng/mL BMP4, and 20 ng/mL FGF2 for 48 hours followed by resting astrocyte media containing only 5 ng/mL HB-EGF for another 72 hours. After this five-day thawing and maturation protocol, experimental treatments could be applied to these mature astrocytes in culture.

Damaging reactive astrocytes were induced in vitro through the exogenous delivery of proinflammatory cytokines 30 ng/mL Tumor Necrosis Factor-alpha (TNFα), 3 ng/mL interleukin 1-alpha (IL1α), and 400 ng/mL complement component 1q (C1q). Exposure of astrocytes to these factors results in the formation of damaging reactive astrocytes that exhibit a cell state specific gene expression signature.

The screening assay to discover chemical compounds capable of blocking the formation of damaging reactive astrocytes is described in more detail below (Table 2).

TABLE 2
Astrocyte culture media conditions.

| | | Resting | Reactive |
|---|---|---|---|
| Astrocyte | Astrocyte | Astrocyte | Astrocyte |
| Enrichment | Maturation | Media | Media |
| 1:1 DMEM/ | 1:1 DMEM/ | 1:1 DMEM/ | 1:1 DMEM/ |
| Neurobasal | Neurobasal | Neurobasal | Neurobasal |
| 2 mM | 2 mM | 2 mM | 2 mM |
| Glutamax | Glutamax | Glutamax | Glutamax |
| 1 mM Sodium | 1 mM Sodium | 1 mM Sodium | 1 mM Sodium |
| Pyruvate | Pyruvate | Pyruvate | Pyruvate |
| 30 μM NAC | 30 μM NAC | 30 μM NAC | 30 μM NAC |
| 1x N2 Max | 1x N2 Max | 1x N2 Max | 1x N2 Max |
| 10 ng/ml | 50 ng/ml | 5 ng/ml | 3 ng/ml |
| BMP4 | BMP4 | HBEGF | IL-1α |
| 10 ng/ml | 10 ng/ml | | 30 ng/ml |
| CNTF | CNTF | | TNFα |
| 5 ng/ml | 5 ng/ml | | 400 ng/ml |
| HBEGF | HBEGF | | C1q |
| 20 ng/ml | 20 ng/ml | | |
| FGF2 | FGF2 | | |

### Primary Screen and Secondary Dose Response Screen.

For screening, all liquid handling was performed using a BioTek EL406 Washer Dispenser. Primary astrocytes were thawed and plated onto 384-well plates as described above at a density of about 500 cells/mm². A Perkin Elmer Janus G3 Varispan Automated Workstation was then used to treat cells with small-molecules with one molecule per well at a concentration of 2 μM in 384-well plates, followed one hour later by 3 ng/mL IL-1α (Sigma #13901), 400 ng/mL C1q (MyBioSource #MBS143105), and 30 ng/mL TNFα (R&D Systems #210-TA-020). After incubation for 24 hours, the cells were fixed using 4% paraformaldehyde and stained for GBP2 (Proteintech #11854-1-AP) using the procedure detailed in the immunocytochemistry section. The cells were then imaged using the PerkinElmer Operetta CLS High-Content Analysis System. Images were analyzed using automated PerkinElmer Columbus Image Analysis Software. For analysis, toxic chemicals were first removed; a chemical was considered toxic if it decreased the counted number of live cells in the well by greater than 30% compared to reactive astrocytes plus DMSO vehicle control wells. Hits were then determined as those compounds that decreased the number of GBP2 positive reactive astrocytes by greater than 80% compared to reactive astrocytes plus DMSO vehicle control wells.

Secondary dose curve screens were performed exactly as described for the primary screen except with custom generated dose curve plates containing the following drug concentrations: 6 μM, 3 μM, 1.5 μM, 0.75 μM, 0.37 μM, 0.18 μM, and 0.09 μM.

### Z-Score Calculation

The standard Z prime (Z′) score is a commonly used measure of drug screen quality. To calculate the standard Z′ where σ=standard deviation and =mean of either the positive or negative control wells:

$$Z ' standard = 1 - \frac{3 {σ}_{positive} + 3 {σ}_{negative}}{\vert {μ}_{positive} - {μ}_{negative} \vert}$$

The robust Z′ is a commonly used measure of drug screen quality that is more robust against data with outliers and is therefore often used for cell based assays where outliers are common. To calculate Z′ Robust where (mad)=mean absolute deviation and x=median of either the positive or negative control wells:

$$Z ' robust = 1 \frac{3 {( mad )}_{p o s i t i v e} + 3 {( m a d )}_{neegatve}}{\vert {\tilde{x}}_{positive} - {\tilde{x}}_{negative} \vert}$$

### Human Astrocyte Generation

Human induced pluripotent stem cells (iPSCs) were cultured to differentiate into astrocytes as described by Perriot et al. (2018). In short, iPSC colonies were placed in neural induction media for 10 days until neural rosettes could be picked, dissociated, and plated in a glial expansion medium. These cells were allowed to proliferate and become a homogenous population of glial progenitor cells (GPCs) over eight passages on poly-L-ornithine and laminin-coated plates. These GPCs were then passaged onto a Matrigel-coated plate to culture in an astrocyte induction media for two weeks, which was followed by culturing for another four weeks in an astrocyte maturation medium.

Real-Time Polymerase Chain Reaction (qPCR)

Cells were lysed in TRIzol and total RNA was extracted with phenol-chloroform followed by spin columns from the RNEasy Mini Kit (74104, QIAGEN). RNA quality and quantity was determined using a NanoDrop spectrophotometer. The RNA was then reverse transcribed using the iScript cDNA synthesis kit (1708891, Bio-Rad) according to the manufacturer's instructions. Real-time qPCR was performed using the Tagman Gene Expression Master Mix (4369016, Applied Biosystems) and the Taqman assay probes for human: GBP2 (Thermo Fisher Assay ID: Hs00894837_ml) and PSMB8 (Hs00544758_ml), and for mouse: (Thermo Fisher Taqman Assay ID: Mm00494576_g1), Psmb8 (Thermo Fisher Taqman Assay ID: Mm00440207_ml), Iigp1 (Thermo Fisher Taqman Assay ID: Mm00649928_s1), and Serping1 (Thermo Fisher Taqman Assay ID: Mm00437835_ml).

### Enzyme-Linked Immunosorbent Assay (ELISA)

Media was collected from mouse P2 primary astrocytes that had been cultured for 24 hrs in the presence of cytokines with and without HDAC3 inhibitors and compared to media from astrocytes not treated with cytokines. The concentration of CCL5, a cytokine secreted by reactive astrocytes, was measured in each of the respective media conditions based on the direct adsorption of CCL5 to a pre-coated capture antibody on the ELISA assay plate. The ELISA sandwich assay was performed according to the manufacturer's instructions (DY478-05, R&D Systems). Relative CCL5 concentration were measured based on absorbance measured by a plate reader.

### OVA₂₅₇₋₂₆₄ Peptide Antigen Presentation Assay

Mouse primary astrocytes were cultured and treated with reactive astrocyte driving cytokines with or without the HDAC3 specific inhibitors RGFP966 and T247. After 24 hours of treatment, these cells were cultured in the presence of the OVA₂₅₇₂₆₄ Peptide (GenScript RP10611 or Sigma S7951). After 12 hours, the cells were live-stained for two hours using a conjugated antibody targeted against an OVA₂₅₇₋₂₆₄ peptide antigen. The cells were then fixed and imaged using the PerkinElmer Operetta CLS High-Content Analysis System. Images were analyzed using the automated Columbus Image Data Storage and Analysis System to identify the extent to which HDAC3 inhibitors decreased OVA₂₅₇₋₂₆₄ antigen presentation.

### NF-κB Jurkat Reporter Assay

NF-κB luciferase reporter Jurkat T-cells were used to measure NF-κB transcriptional activity in response to reactive astrocyte driving cytokines with or HDAC inhibitors and other validated hits from the primary screen. NFκB luciferase reporter Jurkat T-Cells (BPS Biosciences 60651) were purchased from BPS Bioscience. Within this cell line, the firefly luciferase gene is controlled by four copies of an NF-κB response element located upstream of the TATA promoter. Following activation by an external stimulant, endogenous NF-κB transcription factors bound to the DNA response elements to induce transcription of the luciferase gene. Since the luciferase gene is responsible for coding a luminescent protein, the extent of NF-κB signaling pathway activation in the presence of varying doses of the HDAC3 inhibitor, RGFP966, was measured via luminescence using a BioTek Synergy NEO2 plate reader.

### Immunocytochemistry

For immunocytochemistry, cells were fixed with ice-cold 4% PFA for 15 minutes at room temperature, washed three times with PBS, blocked and permeabilized with 10% donkey serum and 0.1% Triton X-100 in PBS for 1 hr, and then stained with primary overnight followed by three washes with PBS, and then 1 hr incubation with Alexaflour secondary antibodies and DAPI. Stained cells were imaged using the PerkinElmer Operetta CLS High-Content Analysis System and analyzed using automated scripts in PerkinElmer Columbus Image Analysis software. Primary antibodies used were GBP2 (ProteinTech, 11854-1-AP), vimentin (BioLegend, 919101), RelA/P65 (Cell Signaling Tech, 6956), MBP (Abeam, AB7349).

### Bulk RNAseq Sample Preparation and Analysis

Total RNA was extracted from resting and reactive astrocytes using the same procedure as described for qPCR and sent to Novogene for mRNA Sequencing.

For gene expression analysis, reads were mapped to the mm10 genome using kallisto 0.46.1 (Bray et al., Nat Biotechnol 34, 525-527, doi:10.1038/nbt.3519 (2016)). Transcripts were summarized to the gene level with tximport (Soneson et al., F1OORes 4, 1521, doi:10.12688/fl000research.7563.2 (2015). Normalized expression and differentiation gene expression was then generated using DESEQ2 (Love et al., Genome Biol 15, 550, doi:10.1186/s13059-014-0550-8 (2014)).

### H3K27Ac and RelA/P65 Chromatin Immunoprecipitation Sequencing (ChIPseq)

Nuclei isolation and chromatin shearing were performed using the Covaris TruChIP protocol following manufacturer's instructions for the “high-cell” format. In brief, 5 million (H3K27Ac) or 20 million resting and reactive (RelA/P65) were crosslinked in “Fixing buffer A” supplemented with 1% fresh formaldehyde for 10 minutes at room temperature with oscillation and quenched for 5 minutes with “Quenchbuffer E.” These cells were then washed with PBS and either snap frozen and stored at 80° C., or immediately used for nuclei extraction and shearing per the manufacturer protocol. The samples were sonicated with the Covaris S2 using the following settings: 5% Duty factor 4 intensity for four 60 s cycles. Sheared chromatin was cleared and incubated overnight at 4 degrees with primary antibodies that were pre-incubated with protein G magnetic DynaBeads (Thermo Fisher, 10004D). Primary antibodies used included anti-H3K27Ac (Abcam, ab4729) and anti-RelA/P65 (Cell Signaling Technology, 8242). These beads were then washed, eluted, reverse cross-linked and treated with RNase A followed by proteinase K. ChIP DNA was purified using Ampure XP beads (Aline Biosciences, C-1003-5) and then used to prepare Illumina sequencing libraries as described previously. Libraries were sequenced on the Illumina HiSeq2500 with single-end 50 bp reads with a read-depth of at least 20 million reads per sample.

For peak calling, reads were quality and adaptors trimmed using Trim Galore!Version 0.4.1. Trimmed reads were aligned to mm10 with Bowtie2 version 2.3.2. Duplicate reads (potential artifacts of PCR in library preparation) were removed using Picard Mark Duplicates. Peaks were called with MACS version 2.1.1. Peaks were visualized with the Integrative Genomics Viewer (IGV, Broad Institute). Peaks were assigned to the nearest gene using bedtools available in Galaxy (Afgan et al., Nucleic Acids Res 46, W537-W544, doi:10.1093/nar/gky379 (2018)).

### Omni Assay for Transposase-Accessible Chromatin Using Sequencing (ATACseq)

Omni ATAC-Seq was performed on 50,000 resting and reactive astrocytes following the protocol outlined in Corces et al. In brief, nuclei were extracted from cells and treated with transposition mixture containing Nextera Tn5 Transposase for (Illumina, FC-121-1030). Transposed fragments were then purified using QIAGEN MinElute columns (QIAGEN, 28004), PCR amplified, and libraries were purified with Agencourt AMPure XP magnetic beads (Aline Biosciences, C-1003-5) with a sample to bead ratio of 1:1.2. Final libraries were sequenced on the Illumina HiSeq2500 with single-end 50 bp reads with nearly 100 million reads per sample. Reads were aligned to the mm10 mouse genome following the same pipeline used for ChIP-seq data (see ChIP-seq and analysis) and peaks were called using the “narrowPeaks” function of MACS version 2.1.1 as outlined for RelA/P65 ChIP-seq (see ChIP-seq and analysis).

### Motif Enrichment Analysis

Motifs were called under RelA/P65 peaks or regions of gained or lost H3K27Ac using HOMERv4.11.1 (Heinz et al., 2010). The FindMotifsGenome.pl tool was used with mm10 as the reference genome.

### Pharmacokinetics

C57BL/6 adult mice were injected i.p. with 10 mg/kg RGFP966 either once followed by tissue collection 4 hrs later, or daily for 11 days. Mice were then perfused with saline to remove blood from the brain. Brain tissue was then collected and snap frozen. Brain tissues were thawed at room temperature and homogenized in PBS. Calibration standards and study samples were extracted with 3× volume of acetonitrile containing 0.1% formic acid+200 ng/ml internal standard solution. Samples were then vortexed each for 1 minute and then transferred to an Eppendorf R5417R and centrifuged at 14000 rpm for 7 minutes. Following extraction of tissue homogenate calibrators and study samples were transferred directly to an autosampler microtiter plate for analysis. Samples were analyzed by LC-MS-MS in the positive ion electrospray ionization mode.

### Lipopolysaccharide (LPS) Model of Neuroinflammation

Mice at 7 weeks of age were injected i.p. with either RGFP966 vehicle (30% hydroxypropyl-β-cyclodextrin, 0.1 M sodium acetate, and 10% DMSO) or 10 mg/kg RGFP966 daily for 11 days. After which mice were injected i.p. daily for two days with either LPS vehicle (saline) plus RGFP966 vehicle, 5 mg/kg LPS plus RGFP966 vehicle, or 5 mg/kg LPS plus 10 mg/kg RGFP966.

### Immunohistochemistry

Mice were perfused with PBS followed by 4% paraformaldehyde, after which brains were extracted and cryopreserved in 30% sucrose then frozen in OCT and sectioned. The stain slides were washed with PBS then incubated overnight with primary antibody against acetyl-Histone H4 (EMD Millipore, 06-866). After primary incubation slides were then washed and labeled with AlexaFluor secondary antibodies (ThermoFisher). Images were captured on a Hamamatsu Nanozoomer S60 Slide scanner with NDP 2.0 software. Image analysis was performed using Perkin Elmer Columbus automated software.

### Lysolecithin (LPC) Injection Model of Focal Tissue Damage

Focal tissue damage in the spinal cord was induced by the injection of 1% LPC solution. 10-12 week old C57BL/6 female mice were anesthetized using isoflurane and a T10 laminectomy was performed. 1 μL of 1% LPC was infused into the dorsal column at a rate of 15 mL/hour. The animals were euthanized at day twelve after the laminectomy (n=6-9 per group). Animals received either RGFP966 vehicle or 10 mg/kg RGFP966 daily by i.p. injection between −1 day post lesion (dpl) to 11 dpl. Mice were then processed for immunohistochemistry or electron microscopy.

In Situ Hybridization with RNAscope

For in vivo studies in situ hybridization was performed using RNAscope Multiplex Fluorescent V2 Assay (ACD Bio, 323136) according to manufacturer's instructions for fixed frozen samples. Briefly, tissue was prepared by first dehydrating with increasingly higher percentages of ethanol, then dried, blocked with hydrogen peroxide, followed by antigen retrieval for 5 minutes, dried again, and then protein was digested using provided Protease III. RNA targeting probes purchased from ACD Bio were then annealed at 40° C. for 2 hrs, followed by washing and a series of amplification steps before finally tagging the RNA with Opal Dye fluorophores (Perkin Elmer). In situ hybridization Images were captured on a Hamamatsu Nanozoomer S60 Slide scanner with NDP 2.0 software. Image analysis was performed using Perkin Elmer Columbus automated software.

### Electron Microscopy Sample Preparation and Analysis

Samples were processed as previously described (Najm et al., Nat Methods 8, 957-962, doi:10.1038/nmeth.1712 (2011)). Briefly, Mice were perfused with 4% PFA, 2% glutaraldehyde, and 0.1 M sodium cacodylate. Spinal cords were extracted and samples were osmicated, stained en bloc with uranyl acetate and embedded in EMbed 812, an Epon-812 substitute (EMS). Thin sections were cut, carbon-coated and imaged either on JEOL JEM-1200-EX electron microscope or a T12 electron microscope (FEI).

### Gene Ontology (GO) and Gene Set Enrichment Analysis (GSEA)

GO analysis was performed using gProfiler (Raudvere et al., Nucleic Acids Res 47, W191-W198, doi:10.1093/nar/gkz369 (2019)) with a significance threshold set at FDR<0.05 and calculated by Benjamin-Hochberg FDR.

GSEA scores were generated for gene sets in the Hallmark v7.4 database using classical scoring, 1000 gene-set permutations, and signal-to-noise metrics. Normalized enrichment scores (NES) and false discovery rate (FDR) were calculated by the GSEA software (Subramanian, A. et al. Gene set enrichment analysis: A knowledge-based approach for interpreting genome-wide expression profiles. Proceedings of the National Academy of Sciences 102, 15545-15550, doi:10.1073/pnas.0506580102 (2005)).

### Statistical Analysis

Unless otherwise noted, GraphPad Prism was used to perform statistical analysis. The statistical tests used, and description of data presentation and sample size can be found in each figure legend.

### Example 7 Pharmacological Inhibition of HDAC3 Suppresses Reactive Astrocytes in vivo

This example demonstrates that treatment with the HDAC3 inhibitor RGFP966 (which has been shown to suppress reactive astrocytes) is neuroprotective in an optic nerve crush model of neurodegeneration.

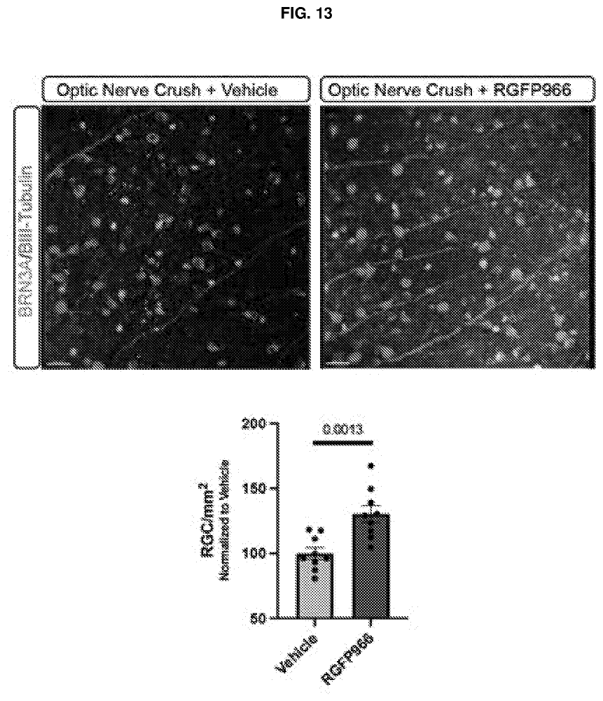

Specifically, FIG. 13 shows that HDAC3 inhibition with RGFP966 protects retinal ganglion cells (RGCs) from neurodegeneration following optic nerve crush. The optic nerve of adult mice was surgically accessed and crushed. Followed by the crush injury, mice were injected daily with either vehicle or 10 mg/kg RGFP966 daily via i.p. for 14 days. 14 days after optic nerve crush surgery, retina were harvested and stained for the RGC marker BRN3A, and the pan neuronal maker BIII-tubulin.

The data in FIG. 13 provides additional evidence that targeting HDACs, and specifically HDAC3, can be used as a treatment for neurodegeneration.

### REFERENCES

1 Bennett, M. L. & Viaene, A. N. What are activated and reactive glia and what is their role in neurodegeneration? Neurobiol Dis 148, 105172 (2021).
2 Liddelow, S. A. et al. Neurotoxic reactive astrocytes are induced by activated microglia. Nature 541, 481-487 (2017).
3 Subramanian, A. et al. Gene set enrichment analysis: A knowledge-based approach for interpreting genome-wide expression profiles. Proceedings of the National Academy of Sciences 102, 15545-15550 (2005).
4 Heinz, S., Romanoski, C. E., Benner, C. & Glass, C. K. The selection and function of cell type-specific enhancers. Nature Reviews Molecular Cell Biology 16, 144-154 (2015).
5 Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Methods 10, 1213-1218 (2013).
6 Shih, R.-H., Wang, C.-Y. & Yang, C.-M. NF-kappaB Signaling Pathways in Neurological Inflammation: A Mini Review. Front Mol Neurosci 8 (2015).
7 Najm, F. J. et al. Drug-based modulation of endogenous stem cells promotes functional remyelination in vivo. Nature 522, 216-220 (2015).
8 Hartmann, K. et al. Complement 3(+)-astrocytes are highly abundant in prion diseases, but their abolishment led to an accelerated disease course and early dysregulation of microglia. Acta Neuropathol Commun 7, 83 (2019).
9 Malvaez, M. et al. HDAC3-selective inhibitor enhances extinction of cocaine-seeking behavior in a persistent manner. Proceedings of the National Academy of Sciences 110, 2647-2652 (2013).
10 Suzuki, T. et al. Identification of Highly Selective and Potent Histone Deacetylase 3 Inhibitors Using Click Chemistry-Based Combinatorial Fragment Assembly. PLoS ONE 8, e68669 (2013).
11 Zamanian, J. L. et al. Genomic analysis of reactive astrogliosis. JNeurosci 32, 6391-6410 (2012).
12 Hao, Y. et al. Integrated analysis of multimodal single-cell data. BioRxiv, 2020.2010.2012.335331 (2020).
13 Bray, N. L., Pimentel, H., Melsted, P. & Pachter, L. Near-optimal probabilistic RNA-seq quantification. Nat Biotechnol 34, 525-527 (2016).
14 Soneson, C., Love, M. I. & Robinson, M. D. Differential analyses for RNA-seq: transcript-level estimates improve gene-level inferences. F1000Res 4, 1521 (2015).
15 Love, M. I., Huber, W. & Anders, S. Moderated estimation of fold change and dispersion for RNA-seq data with DESeq2. Genome Biol 15, 550 (2014).
16 Schmidt, D. et al. ChIP-seq: using high-throughput sequencing to discover protein-DNA interactions. Methods 48, 240-248 (2009).
17 Langmead, B. & Salzberg, S. L. Fast gapped-read alignment with Bowtie 2. Nat Methods 9, 357-359 (2012).
18 Feng, J., Liu, T., Qin, B., Zhang, Y. & Liu, X. S. Identifying ChIP-seq enrichment using MACS. Nat Protoc 7, 1728-1740 (2012).
19 Afgan, E. et al. The Galaxy platform for accessible, reproducible and collaborative biomedical analyses: 2018 update. Nucleic Acids Res 46, W537-W544 (2018).
20 Corces, M. R. et al. An improved ATAC-seq protocol reduces background and enables interrogation of frozen tissues. Nat Methods 14, 959-962 (2017).
21 Najm, F. J. et al. Rapid and robust generation of functional oligodendrocyte progenitor cells from epiblast stem cells. Nat Methods 8, 957-962 (2011).
22 Raudvere, U. et al. g:Profiler: a web server for functional enrichment analysis and conversions of gene lists (2019 update). Nucleic Acids Res 47, W191-W198 (2019).

All sited references and publications are incorporated herein by reference in their entirety, preferably at the instance where they are cited.

1. A method for treating a subject with a disease or disorder associated with reactive astrocytes, comprising administering to the subject a therapeutically effective amount of one or more histone deacetylase (HDAC) inhibitors, wherein the one or more HDAC inhibitors suppress reactive astrocytes (e.g., suppress the formation, maintenance, and/or function of the reactive astrocytes).

2. The method of claim 1, wherein said one or more HDAC inhibitors target HDAC1, HDAC2, HDAC3, HDAC8, or a combination thereof.

3. The method of claim 1, wherein said one or more HDAC inhibitors are specific for HDAC3, such as RGFP966, BRD3308, or HDAC3-IN-T247 (T247).

4. The method of claim 3, wherein the HDAC inhibitor specific for HDAC3 is RGFP966.

5. The method of any one of claims 1-4, wherein the disease or disorder is one or more of disease or disorder of the brain, cerebral injury, brain and systemic disease, neurological disease, cerebral injury, disease associated with loss or reduction of level of calbindin, neurotoxicity, Niemann-Pick disease, Niemann-Pick Type A disease, Niemann-Pick Type B disease, Niemann-Pick Type C disease, neurodegenerative disorder, traumatic brain injury (TBI), autism, Alzheimer's, inflammatory disorder, neuroinflammatory disorder, neuroinflammation due to lysosomal storage disorder, lysosomal storage disorder, Gliobastoma multiforme, HIV, HIV associated cognitive deficits, chemotherapy induced cognitive impairment, immune effector cell-associated neurotoxicity syndrome (ICANS), brain tumor, disease responsive to treatment with histone deacetylase (HDAC) inhibitor, disease involving plasma concentration of vorinostat (SAHA), disease responsive to treatment with SAHA, disease where effect of SAHA is observed in animal model, encephalopathy, epilepsy, cerebrovascular disease, disease responsive to penetration of drug through the blood-brain barrier, Parkinson's disease, Amyotrophic Lateral Sclerosis, activator deficiency/GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, Gaucher's disease, Gaucher disease (types I-III), GM1 gangliosidosis, I-cell disease/muco lipidosis II, infantile free sialic acid storage disease/ISSD, juvenile hexosaminidase A deficiency, Krabbe disease, metachromatic leukodystrophy, mucopolysaccharidoses disorders, pseudo-Hurler polydystrophy/mucolipidosis IIIA, MPSI Hurler syndrome, MPSI Scheie syndrome, MPS I Hurler-Scheie syndrome, MPS II Hunter syndrome, Sanfilippo syndrome, Morquio syndrome, MPS FX hyaluronidase deficiency, MPS VI Maroteaux-Lamy, MPS VII Sly syndrome, mucolipidosis I/sialidosis, multiple sulfatase deficiency, neuronal ceroid lipofuscinoses, Pompe disease, Sandhoff disease, Schindler disease, Salla disease, Tay-Sachs spinal muscular atrophy, Huntington's disease, or a combination thereof.

6. The method of any one of claims 1-4, wherein the disease is selected from the group consisting of a neurodegenerative disease, a myelin or white matter disease, a genetic disease, an inflammatory disease or disorder, an acquired disease or disorder of the central nervous system, and a combination thereof.

7. The method of claim 6, wherein the neurodegenerative disease is Alexander disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, diabetic neuropathy, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), wet or dry macular degeneration, Multiple System Atrophy, multiple sclerosis, Niemann Pick disease, Parkinson's disease, Pelizaeus-Merzbacher Disease, photoreceptor degenerative diseases such as retinitis pigmentosa and associated diseases, Pick's disease, primary lateral sclerosis, prion diseases, Progressive Supranuclear Palsy, Refsum's disease, Sandhoffs disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, or a combination thereof.

8. The method of claim 6, wherein the myelin or white matter disease is acute disseminated encephalomyelitis, destructive leukoencephalopathy, leukoencephalitis, multiple sclerosis, cerebral palsy, periventricular leukomalacia (PVL), hypoxia induced white matter disease, neuromyelitis optica, adult-onset autosomal dominant leukodystrophy (ADLD), Aicardi-Goutieres syndrome, Alexander's disease, CADASIL, Canavan disease, CARASIL, cerebrotendinous xanthomatosis, childhood ataxia and cerebral hypomyelination (CACH)/vanishing white matter disease (VWMD), Fabry disease, fucosidosis, GM1 gangliosidosis, Krabbe's disease, L-2-hydroxyglutaric aciduria, megalencephalic leukoencephalopathy with subcortical cysts, metachromatic leukodystrophy, multiple sulfatase deficiency, Pelizaeus-Merzbacher disease, Pol III-Related Leukodystrophies, Refsum disease, salla disease (free sialic acid storage disease), Sjogren-Larsson syndrome, X-linked adrenoleukodystrophy, Zellweger syndrome spectrum disorders, or a combination thereof.

9. The method of claim 6, wherein the genetic disease is one or more of Huntington's disease, adrenaleukodystrophy, Krabbe's disease, Pelizeaus-Merzbacher disease, vanishing white matter disease, Alexander's disease, metachromatic leukodystrophy, Megalencephalic Leukodystrophy with subcortical Cysts, Canavan's disease, lysosomal storage disorders, leukodystrophies, or a combination thereof.

10. The method of claim 6, wherein the inflammatory disease or disorder is multiple sclerosis, neuromyelitis optica, chronic inflammatory demyelinating polyneuropathy, acute disseminated encephalomyelitis, acute optic neuritis, transverse myelitis, encephalitis, meningitis, or a combination thereof.

11. The method of claim 6, wherein the acquired disease or disorder of the central nervous system is traumatic brain injury, chronic traumatic encephalopathy, stroke, brain metastasis, HIV associated cognitive impairment, neuroaids, cancer related cognitive impairment, immune effector cell-associated neurotoxicity syndrome (ICANS),or a combination thereof.

12. The method of any one of claims 1-4, wherein the disease or disorder is Alzheimer's disease.

13. A method of decreasing, in an subject in need thereof, reactive astrocyte induction/formation/conversion from resting astrocytes, the method comprising: administering to the subject an effective amount of one or more histone deacetylase (HDAC) inhibitors (such as HDAC3-specific inhibitor) to suppress reactive astrocytes; wherein the subject optionally is suspected of having, or at a high risk of having, a disease or disorder associated with reactive astrocytes, and/or has been identified as having reactive astrocytes.

14. The method of claim 13, wherein the subject has been identified as having reactive astrocytes by comparing an image of the brain of the subject with a positive control brain image comprising reactive astrocytes, and a negative control brain image comprising non-reactive or resting astrocytes.

15. The method of claim 13 or 14, wherein the image of the brain of the subject is obtained by positron emission tomography (PET).

16. The method of any one of claims 13-15, wherein said one or more HDAC inhibitors target HDAC1, HDAC2, HDAC3, HDAC8, or a combination thereof.

17. The method of any one of claims 13-15, wherein said one or more HDAC inhibitors are specific for HDAC3, such as RGFP966, BRD3308, or HDAC3-IN-T247 (T247).

18. The method of claim 16, wherein the HDAC inhibitor specific for HDAC3 is RGFP966.

19. The method of any one of claims 13-18, wherein the disease or disorder is a disease or disorder of the brain, cerebral injury, brain and systemic disease, neurological disease, cerebral injury, disease associated with loss or reduction of level of calbindin, neurotoxicity, Niemann-Pick disease, Niemann-Pick Type A disease, Niemann-Pick Type B disease, Niemann-Pick Type C disease, neurodegenerative disorder, traumatic brain injury (TBI), autism, Alzheimer's, inflammatory disorder, neuroinflammatory disorder, neuroinflammation due to lysosomal storage disorder, lysosomal storage disorder, Gliobastoma multiforme, HIV, HIV associated cognitive deficits, non-neurological disease, brain tumor, disease responsive to treatment with histone deacetylase (HDAC) inhibitor, disease involving plasma concentration of vorinostat (SAHA), disease responsive to treatment with SAHA, disease where effect of SAHA is observed in animal model, encephalopathy, epilepsy, cerebrovascular disease, disease responsive to penetration of drug through the blood-brain barrier, Parkinsons, Amyotrophic Lateral Sclerosis, activator deficiency/GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, Gaucher's disease, Gaucher disease (types I-III), GM1 gangliosidosis, I-cell disease/muco lipidosis II, infantile free sialic acid storage disease/ISSD, juvenile hexosaminidase A deficiency, Krabbe disease, metachromatic leukodystrophy, mucopolysaccharidoses disorders, pseudo-Hurler polydystrophy/mucolipidosis IIIA, MPSI Hurler syndrome, MPSI Scheie syndrome, MPS I Hurler-Scheie syndrome, MPS II Hunter syndrome, Sanfilippo syndrome, Morquio syndrome, MPS FX hyaluronidase deficiency, MPS VI Maroteaux-Lamy, MPS VII Sly syndrome, mucolipidosis I/sialidosis, multiple sulfatase deficiency, neuronal ceroid lipofuscinoses, Pompe disease, Sandhoff disease, Schindler disease, Salla disease, Tay-Sachs, spinal muscular atrophy, Huntington's disease, or a combination thereof.

20. The method of any one of claims 13-18, wherein the disease is a neurodegenerative disease, a myelin or white matter disease, a genetic disease, an inflammatory disease or disorder, an acquired disease or disorder of the central nervous system, or a combination thereof.

21. The method of claim 20, wherein the neurodegenerative disease is one or more of Alexander disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, diabetic neuropathy, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), wet or dry macular degeneration, Multiple System Atrophy, multiple sclerosis, Niemann Pick disease, Parkinson's disease, Pelizaeus-Merzbacher Disease, photoreceptor degenerative diseases such as retinitis pigmentosa and associated diseases, Pick's disease, primary lateral sclerosis, prion diseases, Progressive Supranuclear Palsy, Refsum's disease, Sandhoffs disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, or a combination thereof.

22. The method of claim 20, wherein the myelin or white matter disease is acute disseminated encephalomyelitis, destructive leukoencephalopathy, leukoencephalitis, multiple sclerosis, cerebral palsy, periventricular leukomalacia (PVL), hypoxia induced white matter disease, neuromyelitis optica, adult-onset autosomal dominant leukodystrophy (ADLD), Aicardi-Goutieres syndrome, Alexander's disease, CADASIL, Canavan disease, CARASIL, cerebrotendinous xanthomatosis, childhood ataxia and cerebral hypomyelination (CACH)/vanishing white matter disease (VWMD), Fabry disease, fucosidosis, GM1 gangliosidosis, Krabbe's disease, L-2-hydroxyglutaric aciduria, megalencephalic leukoencephalopathy with subcortical cysts, metachromatic leukodystrophy, multiple sulfatase deficiency, Pelizaeus-Merzbacher disease, Pol III-Related Leukodystrophies, Refsum disease, salla disease (free sialic acid storage disease), Sjogren-Larsson syndrome, X-linked adrenoleukodystrophy, Zellweger syndrome spectrum disorders, or a combination thereof.

23. The method of claim 20, wherein the genetic disease is Huntington's disease, adrenaleukodystrophy, Krabbe's disease, Pelizeaus-Merzbacher disease, vanishing white matter disease, Alexander's disease, metachromatic leukodystrophy, Megalencephalic Leukodystrophy with subcortical Cysts, Canavan's disease, lysosomal storage disorders, leukodystrophies, or a combination thereof.

24. The method of claim 20, wherein the inflammatory disease or disorder is multiple sclerosis, neuromyelitis optica, chronic inflammatory demyelinating polyneuropathy, acute disseminated encephalomyelitis, acute optic neuritis, transverse myelitis, encephalitis, meningitis, or a combination thereof.

25. The method of claim 20, wherein the acquired disease or disorder of the central nervous system is traumatic brain injury, chronic traumatic encephalopathy, stroke, brain metastasis, neuroaids, cancer related cognitive impairment, immune effector cell-associated neurotoxicity syndrome (ICANS), or a combination thereof.

26. The method of any one of claims 13-18, wherein the disease or disorder is Alzheimer's disease.

27. A method of inhibiting RelA/P65-mediated transcription in a reactive astrocyte, the method comprising: contacting the reactive astrocyte with an effective amount of one or more histone deacetylase (HDAC) inhibitors (such as HDAC3-specific inhibitor).

28. The method of claim 27, wherein said HDAC inhibitors inhibit nuclear translocation of RelA/P65.

29. A method of decreasing very long chain fatty acids (VLCFA) in a reactive astrocyte, the method comprising: contacting the reactive astrocyte with an effective amount of one or more histone deacetylase (HDAC) inhibitors (such as HDAC3-specific inhibitor).

30. The method of claim 28, wherein the HDAC inhibitors inhibit VLCFA production.

31. The method of and one of claims 27-30, wherein said one or more HDAC inhibitors target HDAC1, HDAC2, HDAC3, HDAC8, or a combination thereof.

32. The method of and one of claims 27-30, wherein said one or more HDAC inhibitors are specific for HDAC3, such as RGFP966, BRD3308, or HDAC3-IN-T247 (T247).

33. The method of claim 32, wherein the HDAC inhibitor specific for HDAC3 is RGFP966.

34. The method of any one of claims 27-33, wherein the reactive astrocyte is in a subject suspected of having, or at a high risk of having, a disease or disorder associated with reactive astrocytes, and/or has been identified as having reactive astrocytes.

35. A method of promoting CNS tissue repair in a subject in need thereof, the method comprising: administering to the subject a therapeutically effective amount of one or more histone deacetylase (HDAC) inhibitors, wherein the one or more HDAC inhibitors suppress reactive astrocytes (e.g., suppress the formation, maintenance, and/or function of the reactive astrocytes, such as GBP2⁺ reactive astrocytes).

36. The method of claim 35, wherein said CNS tissue has neuronal demyelination, and/or axonal damage, and wherein said CNS tissue repair comprises axonal remyelination.

37. The method of claim 35 or 36, wherein said one or more HDAC inhibitors target HDAC1, HDAC2, HDAC3, HDAC8, or a combination thereof.

38. The method of claim 35 or 37, wherein said one or more HDAC inhibitors are specific for HDAC3, such as RGFP966, BRD3308, or HDAC3-IN-T247 (T247).

39. The method of claim 38, wherein the HDAC inhibitor specific for HDAC3 is RGFP966.
